(19) United States
(12) Patent Application Publication
(10) Pub. No.: US 2017/0100396 A1
(43) Pub. Date: Apr. 13, 2017

(54) PYRROLOPYRAZINE DERIVATIVES FOR USE IN THE TREATMENT, AMELIORATION OR PREVENTION OF INFLUENZA

(71) Applicants: F. Hoffmann-La Roche AG, Basel (CH); Savira pharmaceuticals GmbH, Vienna (AT); European Molecular Biology Laboratory, Heidelberg (DE)

(72) Inventors: Tanja SCHULZ-GASCH, Ziefen (CH); Robert WEIKERT, Basel (CH); Werner NEIDHART, Basel (CH); Andrea WOLKERSTORFER, Vienna (AT); Oliver SZOLAR, Vienna (AT); Norbert HANDLER, Vienna (AT); Helmut BUSCHMANN, Aachen (DE); Stephen CUSACK, Seyssinet-Pariset (FR)

(21) Appl. No.: 15/285,847

(22) Filed: Oct. 5, 2016

Related U.S. Application Data
(60) Provisional application No. 62/238,415, filed on Oct. 7, 2015.

(51) Int. Cl. A61K 31/4985; A61K 31/5377; A61K 31/541; A61K 45/06; A61K 31/506

(52) U.S. Cl. CPC A61K 31/4985; A61K 45/06; A61K 31/506; A61K 31/541; A61K 31/5377

(57) ABSTRACT

The present invention relates to a compound having the general formula (I), optionally in the form of a pharmaceutically acceptable salt, solvate, polymorph, prodrug, tautomer, racemate, codrug, cocrystal, enantiomer, or diastereomer or mixture thereof,

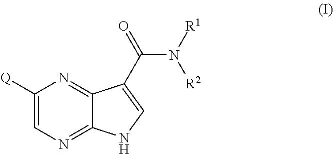

which is useful in treating, ameliorating or preventing influenza. Furthermore, specific combination therapies are disclosed.

### CROSS-REFERENCE TO RELATED APPLICATIONS

This application claims the benefit of U.S. Provisional Patent Application No. 62/238,415, filed Oct. 7, 2015, which is incorporated herein by reference in its entirety.

### FIELD OF THE INVENTION

The present invention relates to a compound having the general formula (I), optionally in the form of a pharmaceutically acceptable salt, solvate, polymorph, prodrug, tautomer, racemate, codrug, cocrystal, enantiomer, or diastereomer or mixture thereof,

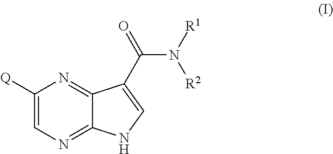

which is useful in treating, ameliorating or preventing influenza. Furthermore, specific combination therapies are disclosed.

### BACKGROUND OF THE INVENTION

In recent years the serious threat posed by influenza virus to worldwide public health has been highlighted by, firstly, the ongoing low level transmission to humans of the highly pathogenic avian H5N1 strain (63% mortality in infected humans, http://www.who.int/csr/disease/avian_influenza/en/) and secondly, the unexpected emergence in 2009 of a novel pandemic strain A/H1N1 that has rapidly spread around the entire world (http://www.who.int/csr/disease/swineflu/en/). Whilst the new strain is highly contagious but currently generally only gives mild illness, the future evolution of this virus is unpredictable. In a much more serious, but highly plausible scenario, H5N1 could have been more easily transmissible between humans or the new A/H1N1 could have been more virulent and could have carried the single point mutation that confers Tamiflu resistance (Neumann et al., Nature, 2009 (18; 459(7249) 931-939), as many seasonal H1N1 strains have recently done (Dharan et al., The Journal of the American Medical Association, 2009 Mar. 11; 301 (10), 1034-1041; Moscona et al., The New England Journal of Medicine, 2009 (March 5; 360(10) pp 953-956). In this case, the delay in generating and deploying a vaccine (˜6 months in the relatively favorable case of A/H1N1 and still not a solved problem for H5N1) could have been catastrophically costly in human lives and societal disruption.

It is widely acknowledged that to bridge the period before a new vaccine becomes available and to treat severe cases, as well as to counter the problem of viral resistance, a wider choice of anti-influenza drugs is required. Development of new anti-influenza drugs has therefore again become a high priority, having been largely abandoned by the major pharmaceutical companies once the anti-neuraminidase drugs became available.

An excellent starting point for the development of antiviral medication is structural data of essential viral proteins. Thus, the crystal structure determination of e.g. the influenza virus surface antigen neuraminidase (Von Itzstein, M. et al., (1993), Nature, 363, pp. 418-423) led directly to the development of neuraminidase inhibitors with anti-viral activity preventing the release of virus from the cells, however, not the virus production. These and their derivatives have subsequently developed into the anti-influenza drugs, zanamivir (Glaxo) and oseltamivir (Roche), which are currently being stockpiled by many countries as a first line of defence against an eventual pandemic. However, these medicaments only provide a reduction in the duration of the clinical disease. Alternatively, other anti-influenza compounds such as amantadine and rimantadine target an ion channel protein, i.e., the M2 protein, in the viral membrane interfering with the uncoating of the virus inside the cell. However, they have not been extensively used due to their side effects and the rapid development of resistant virus mutants (Magden, J. et al., (2005), Appl. Microbiol. Biotechnol., 66, pp. 612-621). In addition, more unspecific viral drugs, such as ribavirin, have been shown to work for treating of influenza and other virus infections (Eriksson, B. et al., (1977), Antimicrob. Agents Chemother., 11, pp. 946-951). However, ribavirin is only approved in a few countries (Furuta et al., Antimicrobial Agents and Chemotherapy, 2005 March 49(3); 981-986), probably due to severe side effects. Clearly, new antiviral compounds are needed, preferably directed against different targets.

Influenza viruses are negative stranded RNA viruses. Their genome is segmented and comes in ribonucleoprotein particles that include the RNA dependent RNA polymerase which carries out (i) the initial copying of the single-stranded virion RNA (vRNA) into viral mRNAs and (ii) the vRNA replication. This enzyme, a trimeric complex composed of subunits PA, PB1 and PB2, is central to the life cycle of the virus since it is responsible for the replication and transcription of viral RNA. In previous work the atomic structure of two key domains of the polymerase, the mRNA cap-binding domain in the PB2 subunit (Guilligay et al., Antimicrobial Agents and Chemotherapy, 2005 March 49(3); pp 981-986) and the endonuclease-active site in the PA subunit (Dias et al., Nature 2009; April 16; 458(7240); 914-918) have been identified and determined. These two sites are critical for the unique cap-snatching mode of transcription that is used by influenza virus to generate viral mRNAs. For the generation of viral mRNA the polymerase makes use of the so called “cap-snatching” mechanism (Plotch, S. J. et al., (1981), Cell, 23, pp. 847-858; Kukkonen, S. K. et al (2005), Arch. Virol., 150, pp. 533-556; Leahy, M. B. et al, (2005), J. Virol., 71, pp. 8347-8351; Noah, D. L. et al., (2005), Adv. Virus Res., 65, pp. 121-145). A 5′ cap (also termed an RNA cap, RNA 7-methylguanosine cap or an RNA m7G cap) is a modified guanine nucleotide that has been added to the 5′ end of each cellular messenger RNA. The 5′RNA cap consists of a terminal 7-methylguanosine residue which is linked through a 5′-5′-triphosphate bond to the first transcribed nucleotide. Upon influenza virus infection the 5′RNA cap of cellular mRNA molecules is bound by the viral polymerase complex, specifically the cap-binding domain within the PB2 subunit of the polymerase complex, and the RNA cap together with a stretch of 10 to 15 nucleotides is cleaved by the viral endonuclease which resides within the PA subunit of the viral polymerase complex. The capped RNA fragments then serve as primers for the synthesis of viral mRNA.

The cap-binding domain in the PB2 subunit of the viral polymerase has been unequivocally identified and structurally characterized by Guilligay et al., 2008. Binding the capped host cell mRNA via the cap-binding site and hence bringing the host cell mRNA strand into close spatial vicinity of the endonuclease active site is a prerequisite for the endonuclease to snatch off the cap. Therefore the cap-binding site in PB2 is essential for cap-dependent transcription by the viral RNPs and mandatory for the viral replication cycle. This together with the fact that the PB2 cap-binding domain is structurally distinct from other cap binding proteins, this suggests that the ligand binding site is a good target for the development of new antiviral drugs.

Generally, the polymerase complex seems to be an appropriate antiviral drug target since it is essential for synthesis of viral mRNA and viral replication and contains several functional active sites likely to be significantly different from those found in host cell proteins (Magden, J. et al., (2005), Appl. Microbiol. Biotechnol., 66, pp. 612-621). Thus, for example, there have been attempts to interfere with the assembly of polymerase subunits by a 25-amino-acid peptide resembling the PA-binding domain within PB1 (Ghanem, A. et al., (2007), J. Virol., 81, pp. 7801-7804). Furthermore, the endonuclease activity of the polymerase has been targeted and a series of 4-substituted 2,4-dioxobutanoic acid compounds has been identified as selective inhibitors of this activity in influenza viruses (Tomassini, J. et al., (1994), Antimicrob. Agents Chemother., 38, pp. 2827-2837). In addition, flutimide, a substituted 2,6-diketopiperazine, identified in extracts of Delitschia confertaspora, a fungal species, has been shown to inhibit the endonuclease of influenza virus (Tomassini, J. et al., (1996), Antimicrob. Agents Chemother., 40, pp. 1189-1193). Moreover, there have been attempts to interfere with viral transcription by nucleoside analogs, such as 2′-deoxy-2′-fluoroguanosine (Tisdale, M. et al., (1995), Antimicrob. Agents Chemother., 39, pp. 2454-2458).

WO 2009/106441, WO2009/106442, WO 2009/106443; WO 2009/106444; WO 2009/106445; WO 2011/117145, WO 2011/117160, WO 2011/144584, and WO 2011/144585 disclose certain pyrrolopyrazine derivatives.

It is an object of the present invention to identify compounds which specifically target the influenza virus cap-binding domain and hence are effective against influenza and which have improved pharmacological properties.

### SUMMARY OF THE INVENTION

Accordingly, in a first embodiment, the present invention provides a compound having the general formula (I) for use in the treatment, amelioration or prevention of influenza.

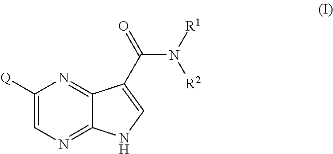

It is understood that throughout the present specification the term “a compound having the general formula (I)” encompasses pharmaceutically acceptable salts, solvates, polymorphs, prodrugs, tautomers, racemates, codrug, cocrystal, enantiomers, or diastereomers or mixtures thereof unless mentioned otherwise.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### DEFINITIONS

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps.

When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbol “ [symbol] ” at the end of a bond refers to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The phrase “come together to form a ring” as used herein means join to form a ring, wherein the ring may be made up of either 4-7 carbon atoms or 4-7 carbon and heteroatoms, and may be saturated or unsaturated.

The phrase “come together to form a bicyclic ring system” as used herein means join to form a bicyclic ring system, wherein each ring may be made up of either 4-7 carbon atoms or 4-7 carbon and heteroatoms, and may be saturated or unsaturated.

The term “about” is used herein to mean approximately, in the region of, roughly, or around. When the term “about” is used in conjunction with a numerical range, it modifies that range by extending the boundaries herein and below the numerical values set forth. In general, the term “about” is used herein to modify a numerical value herein above and below the stated value by a variance of 20%.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” “cycloalkylalkyl” and the like. When the term “alkyl” is used as a suffix following another term, as in “phenylalkyl,” or “hydroxyalkyl,” this is intended to refer to an alkyl group, as defined herein, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, I-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

Compounds having the formula (I) may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH— [symbol] —C(—OH)═CH—), amide/imidic acid (—C(═O)—NH— [symbol] —C(—OH)═N—) and amidine (—C(═NR)—NH— [symbol] —C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

As used herein, the term “organic substituent” comprises any substituent comprising carbon and in addition also comprises hydrogen and halogens.

The term “acyl” as used herein denotes a group of formula —C(═O)R wherein R is hydrogen or lower alkyl as defined herein.

The term or “alkylcarbonyl” as used herein denotes a group of formula —C(═O)R wherein R is alkyl as defined herein. The term C₁₋₆ acyl refers to a group —C(═O)R contain 6 carbon atoms. The term “arylcarbonyl” as used herein means a group of formula —C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

The term “alkenyl” as used herein denotes an unbranched or branched chain, monovalent hydrocarbon residue containing 2 to 10 carbon atoms which includes at least one double bond. The term “lower alkenyl” denotes a straight or branched chain hydrocarbon residue containing 2 to 6 carbon atoms which includes at least one double bond. “C₂₋₁₀ alkenyl” as used herein refers to an alkenyl composed of 2 to 10 carbons.

The term “alkynyl” as used herein denotes an unbranched or branched chain, monovalent hydrocarbon residue containing 2 to 10 carbon atoms which includes at least one triple bond. The term “lower alkynyl” denotes a straight or branched chain hydrocarbon residue containing 2 to 6 carbon atoms which includes at least one triple bond. “C₂₋₁₀ alkenyl” as used herein refers to an alkenyl composed of 2 to 10 carbons.

When the term “alkyl” is used as a suffix following another term, as in “phenylalkyl” or “hydroxyalkyl”, this is intended to refer to an alkyl group, as defined herein, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl”, “aryl alkyl”, or “aralkyl” are interpreted similarly except R′ is an aryl radical. The terms “heteroaryl alkyl” or “heteroarylalkyl” are interpreted similarly except R′ is optionally an aryl or a heteroaryl radical.

The term “haloalkyl” as used herein denotes a unbranched or branched chain alkyl group as defined herein wherein 1, 2, 3 or more hydrogen atoms are substituted by a halogen. The term “lower haloalkyl” denotes a straight or branched chain hydrocarbon residue containing 1 to 6 carbon atoms, wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “alkylene” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, and 2-ethylbutylene.

The term “alkoxy” as used herein means an —O-alkyl group, wherein alkyl is as defined herein such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “alkoxyalkyl” as used herein refers to the radical R′R″-, wherein R′ is an alkoxy radical as defined herein, and R″ is an alkylene radical as defined herein with the understanding that the attachment point of the alkoxyalkyl moiety will be on the alkylene radical. C₁₋₆ alkoxyalkyl denotes a group wherein the alkyl portion is comprised of 1-6 carbon atoms exclusive of carbon atoms in the alkoxy portion of the group. C₁₋₃ alkoxy-C₁₋₆ alkyl denotes a group wherein the alkyl portion is comprised of 1-6 carbon atoms and the alkoxy group is 1-3 carbons. Examples are methoxymethyl, methoxyethyl, methoxypropyl, ethoxymethyl, ethoxyethyl, ethoxypropyl, propyloxypropyl, methoxybutyl, ethoxybutyl, propyloxybutyl, butyloxybutyl, t-butyloxybutyl, methoxypentyl, ethoxypentyl, propyloxypentyl including their isomers.

The term “hydroxyalkyl” as used herein denotes an alkyl radical as herein defined wherein one to three hydrogen atoms on different carbon atoms is/are replaced by hydroxyl groups.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term “cycloalkenyl” refers to a partially unsaturated carbocyclic containing 5 to 7 carbon atoms unless otherwise specified and having a carbon-carbon double bond within the ring. For example, C₅₋₆ cycloalkenyl refers to a cycloalkenyl group having from 5 to 6 member atoms. In certain embodiments cycloalkenyl groups have one carbon-carbon double bond within the ring. In other embodiments, cycloalkenyl groups have more than one carbon-carbon double bond within the ring. However, cycloalkenyl rings are not aromatic. Cycloalkenyl groups may be optionally substituted with one or more substituent. Examples of cycloalkenyl include, but are not limited to, cyclopentenyl and cyclohexenyl.

The term “halogen” or “halo” as used herein means fluorine, chlorine, bromine, or iodine.

The term “amino” as used herein encompasses —NR₂, wherein each R group is independently H or lower alkyl, wherein lower alkyl is as defined herein. Examples of amino groups include dimethyl amino, methyl amino and NH₂.

As used herein, the term “aryl” means a monocyclic or bicyclic (also referred to as “biaryl”), substituted or unsubstituted carbocyclic aromatic group. Examples of aryl groups are phenyl, naphthyl and the like.

The term “heteroaryl” or “heteroaromatic” as used herein means a monocyclic, bicyclic, or tricyclic radical of 5 to 18 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole.

The term “heteroaryloxy” as used herein means an —O-(heteroaryl) group wherein heteroaryl is defined herein.

The term (hetero)aryl as used herein refers to an aryl or a heteroaryl moiety as each is defined herein.

The term “heterocycloalkyl”, “heterocyclyl” or “heterocycle” as used herein denotes a monovalent saturated cyclic radical, consisting of one or more rings, preferably one to two rings or three rings, of three to eight atoms per ring, incorporating one or more ring carbon atoms and one or more ring heteroatoms (chosen from N, O or S(═O)₀₋₂), wherein the point of attachment can be through either a carbon atom or a heteroatom, and which can optionally be independently substituted with one or more, preferably one or two or three substituents selected from hydroxy, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

The term “heterocycloalkyloxy” as used herein means an —O-(heterocycloalkyl) group wherein heterocycloalkyl is defined herein.

The term “heteroatom containing moieties” as used herein means moieties which contain heteroatoms such as N, O or S. The heteroatom containing moieties include —C(O)—, —C(O)—NH—, —C(O)—O— and the like.

If a compound or moiety is referred to as being “optionally substituted” it can in each instance include 1 or more of the indicated substituents, whereby the substituents can be the same or different.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use. A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Suitable pharmaceutically acceptable salts include acid addition salts which may, for example, be formed by mixing a solution of compounds of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)).

When the compounds of the present invention are provided in crystalline form, the structure can contain solvent molecules. The solvents are typically pharmaceutically acceptable solvents and include, among others, water (hydrates) or organic solvents. Examples of possible solvates include ethanolates and iso-propanolates.

The term “codrug” refers to two or more therapeutic compounds bonded via a covalent chemical bond. A detailed definition can be found, e.g., in N. Das et al., European Journal of Pharmaceutical Sciences, 41, 2010, 571-588.

The term “cocrystal” refers to a multiple component crystal in which all components are solid under ambient conditions when in their pure form. These components co-exist as a stoichiometric or non-stoichometric ratio of a target molecule or ion (i.e., compound of the present invention) and one or more neutral molecular cocrystal formers. A detailed discussion can be found, for example, in Ning Shan et al., Drug Discovery Today, 13(9/10), 2008, 440-446 and in D. J. Good et al., Cryst. Growth Des., 9(5), 2009, 2252-2264.

The compounds of the present invention can also be provided in the form of a prodrug, namely a compound which is metabolized in vivo to the active metabolite.

### Compounds Having the General Formula (I)

The compounds useful in the present invention have the general formula (I):

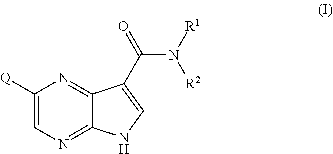

These compounds are known from WO 2009/106441, WO2009/106442, WO 2009/106444; WO 2011/117145, WO 2011/117160, WO 2011/144584, and WO 2011/144585. These references, in particular, the description of the compounds having the general formula (I), the definitions of the moieties Q, R¹ and R² (including the preferred definitions thereof), the exemplified compounds as well as the methods for the preparation of the compounds, are incorporated herein in their entirety by reference.

In the above general formula (I)

Q is an organic substituent;

R¹ is an organic substituent; and

R² is an organic substituent.

In a preferred embodiment, Q is Q¹, Q², or Q³; more preferably Q is Q₁.

In a preferred embodiment, Q¹ is cycloalkyl, heterocycloalkyl, cycloalkyloxy, cycloalkenyl, heterocycloalkyl aryl, aryloxy, heteroaryl, biaryl, or heterobiaryl, optionally substituted with one or more Q^{1a};

Q^{1a} is Q^{1b} Or Q^{1c};

each Q^{1b} is independently halogen, oxo, hydroxy, cyano, —SCH₃, —S(O)₂CH₃, or —S(═O)CH₃;

each Q^{1c} is independently Q^{1d} or Q^{1e};

or two Q^{1a} come together to form a bicyclic ring system, optionally substituted with one or more Q^{1b} or Q^{1c};

each Q^{1d} is independently —O(Q₁ₑ), —S(═O)₂(Q^{1e}), —C(═O)N(Q^{1e})₂, —S(O)₂(Q^{1e}), —C(═O)(Q₁ₑ), —C(═O)O(Q^{1e}), —N(Q^{1e})₂, —N(Q^{1e})C(═O)(Q^{1e}), —N(Q^{1e})C(═O)O(Q^{1e}), or —N(Q^{1e})C(═O)N(Q^{1e})₂;

each Q^{1e} is independently H or Q^{1j};

each Q^{1f} is independently Q^{1g} or Q^{1h};

each Q^{1g} is independently halogen, hydroxy, cyano, oxo, —C(═O)(Q^{1h}), —S(═O)₂(Q^{1k}), —S(═O)₂N(Q^{1k})₂, —C(═O)OH, C(═O)N(Q^{1k})₂, or —C(═O)(Q^{1k});

each Q^{1h} is independently lower alkyl, lower alkenyl, lower haloalkyl, lower alkoxy, amino, aryl, benzyl, cycloalkyl, heterocycloalkyl, or heteroaryl, optionally substituted with one or more Q¹ⁱ;

each Q^{1l} is independently halogen, hydroxy, cyano, lower alkyl, lower haloalkyl, or lower alkoxy;

each Q^{1j} is independently lower alkyl, aryl, benzyl, 5,6,7,8-tetrahydro-naphthalene, lower haloalkyl, lower alkoxy, cycloalkyl, cycloalkyl lower alkyl, cycloalkenyl, heterocycloalkyl, spirocyclic heterocycloalkyl, or heteroaryl, optionally substituted with one or more Q^{1f};

each Q^{1k} is independently H or lower alkyl.

In a preferred embodiment, Q² is Q^{2a} or Q^{2b};

Q^{2a} is H, hydroxy, halogen, or cyano;

Q^{2b} is lower alkyl, lower alkoxy, lower alkenyl, lower alkynyl, lower hydroxyalkyl, amino, or lower haloalkyl, optionally substituted with one or more Q^{2c};

Q^{2c} is Q^{2d} or Q^{2e};

Q^{2d} is halogen, oxo, hydroxy, cyano, —C(═O)(Q^{2j}), —SCH₃, —S(O)₂CH₃, or —S(═O)CH₃;

Q^{2e} is Q^{2f} Or Q^{2j};

or two Q^{2c} come together to form a bicyclic ring system, optionally substituted with one or more Q^{2d} or Q^{2e};

Q^{2f} is —O(Q^{2g}), —S(═O)₂(Q^{2g}), —C(═O)N(Q^{2g})₂, —S(O)₂(Q^{2g}), —C(═O)(Q^{2g}), —C(═O)O(Q^{2g}), —N(Q^{2g})₂; —N(Q^{2g})C(═O)(Q^{2g})-N(Q^{2g})C(═O)O(Q^{2g}), or —N(Q^{2g})C(═O)N(Q^{2g})₂;

each Q^{2g} is independently H or Q^{2m};

Q^{2h} is Q²ⁱ or Q^{2j};

Q²ⁱ is halogen, hydroxy, cyano, oxo, or —C(═O)(Q²);

Q^{2j} is lower alkyl, lower alkenyl, lower alkoxy, amino, aryl, benzyl, cycloalkyl, heterocycloalkyl, or heteroaryl, optionally substituted with one or more Q^{2k};

Q^{2k} is halogen, hydroxy, cyano, lower alkyl, lower haloalkyl, lower alkenyl, oxo, lower hydroxyalkyl, amino or lower alkoxy;

each Q^{2m} is independently lower alkyl, aryl, benzyl, lower haloalkyl, lower alkoxy, amino, cycloalkyl, cycloalkyl lower alkyl, cycloalkenyl, heterocycloalkyl, or heteroaryl, optionally substituted with one or more Q^{2h}.

In a preferred embodiment, Q³ is aryl or heteroaryl, optionally substituted with one or more Q^{3a};

each Q^{3a} is independently Q^{3b} or Q^{3c};

each Q^{3b} is independently halogen, hydroxy, cyano, —S(Q^{3e}), —S(O)₂(Q^{3e}), or —S(═O)(Q^{3e});

each Q^{3c} is independently Q^{3d} or Q^{3e};

each Q^{3d} is independently —O(Q^{3e}), —S(═O)₂(Q^{3e}), —C(═O)N(Q^{3e})₂, —S(═O)(Q^{3e}), —N(Q^{3e})S(═O)₂(Q^{3e}), —C(═O)(Q^{3e}), —C(═O)O(Q^{3e}), —N(Q^{3e})₂, —N(Q^{3e})C(═O)(Q^{3e}), —N(Q^{3e})C(═O)O(Q^{3e}), —Si(Q^{3e})₃, or —N(Q^{3e})C(═O)N(Q^{3e})₂;

each Q^{3e} is independently H or Q^{3m};

each Q^{3f} is independently Q^{3g} or Q^{3h};

each Q^{3g} is independently halogen, hydroxy, oxo, —(C(Q^{3h})₂)_{mQ}S(O)₂(Q^{3h}), —(C(Q^{3h})₂)_{mQ}N(Q^{3h})(C(Q^{3h})₂)_{mQ}S(O)₂(Q^{3h}), —(C(Q^{3h})₂)_{mQ}N(Q^{3h})₂, —(C(Q^{3h})₂)_{mQ}C(═O)(Q^{3h}), or —N(Q^{3h})C(═O)(Q^{3h});

each Q^{3h} is independently Q³ⁱ Or Q^{3j};

each Q³ⁱ is independently H or hydroxy;

each Q^{3j} is independently lower alkyl, lower haloalkyl, lower alkoxy, lower thioalkyl, cyano, amino, aryl, benzyl, cycloalkyl, heterocycloalkyl, or heteroaryl, optionally substituted with one or more Q^{3k};

each Q^{3k} is independently halogen, hydroxy, lower alkyl, lower haloalkyl, lower hydroxyalkyl, amino, lower thioalkyl, lower alkoxy, or cyano;

each Q^{3m} is independently lower alkyl, amino, lower alkenyl, aryl, benzyl, lower haloalkyl, lower thioalkyl, cycloalkyl, cycloalkenyl, heterocycloalkyl, heterocycloalkyl alkylene, or heteroaryl, optionally substituted with one or more Q^{3f}.

In a preferred embodiment, Q is selected from the group consisting of cycloalkyl, halogen, lower alkyl and aryl which is optionally substituted with one or more Q^{3a}, wherein Q^{3a} is selected from the group consisting of halogen, haloalkyl, cycloalkyl-C(O)—OH and cycloalkyl-C(O)—O-(lower alkyl).

In a preferred embodiment, Q is selected from the group consisting of cycloalkyl and aryl which is optionally substituted with cycloalkyl-C(O)—OH or cycloalkyl-C(O)—O-(lower alkyl).

In a more preferred embodiment, Q is cyclopropyl.

Each m_{Q} is preferably independently 0, 1, or 2.

Preferably R¹ and R² are selected from (i) to (v). In one embodiment, R¹ and R² are as defined in embodiment (i). In one embodiment, R¹ and R² are as defined in embodiment (ii). In one embodiment, R¹ and R² are as defined in embodiment (iii). In one embodiment, R¹ and R² are as defined in embodiment (iv). In one embodiment, R¹ and R² are as defined in embodiment (v).

In embodiment (i), R¹ is H and

R² is —Y—C(O)—NR^{1e}R^{1g};

Y is C(R^{1a})₂(C(R^{1b})₂)m_{R};

m_{R} is 0 or 1;

each R^{1a} is H or R^{1c};

each R^{1b} is independently H, lower alkyl, lower haloalkyl, aryl, heteroaryl, cycloalkyl, heterocycloalkyl, wherein the aryl, heteroaryl, cycloalkyl, heterocycloalkyl can be optionally substituted by H, halogen, lower alkyl, lower alkoxy, or lower haloalkyl;

each R^{1c} is independently lower alkyl, lower alkoxy, aryl, benzyl, heteroaryl, cycloalkyl, heterocycloalkyl, or cycloalkyl lower alkyl, optionally substituted with one or more R^{1d};

R^{1d} is independently R^{1j} or R^{1k};

R^{1e} is independently H or R^{1f};

R^{1f} is independently lower alkyl, lower alkoxy, aryl, heteroaryl, cycloalkyl, heterocycloalkyl, bicyclic ring system or spirocyclic ring system, wherein the bicyclic ring system or spirocyclic ring system can optionally include one or more heteroatoms or heteroatom containing moieties such as C═O, wherein R^{1f} can be optionally substituted with one or more R^{1d};

or R^{1f} and R^{1c} come together to form a ring, optionally substituted with one or more one or more halogen, lower alkyl, cyano, cyano lower alkyl, hydroxy, lower haloalkyl, lower hydroxyalkyl, lower alkoxy, lower alkylamino, or lower dialkylamino;

R^{1g} is independently H or R^{1h};

R^{1h} is independently lower alkyl, lower haloalkyl, lower alkoxy, lower hydroxyalkyl, cyano lower alkyl, C(═O)R¹ⁱ or S(═O)₂R¹ⁱ;

each R¹ⁱ is independently H or lower alkyl;

R^{1j} is independently halogen, lower alkyl, lower haloalkyl, lower alkoxy, lower haloalkoxy, lower hydroxyalkyl, oxo, hydroxy, C(═O)—NH—(CH₂)ₙ₁—R^{1b}, C(═O)—(CH₂)ₙ₁—R^{1b}, (C═O)—OR^{1b} or cyano;

R^{1k} is independently —(CH₂)ₙ₁-cycloalkyl, —(CH₂)ₙ₁-heterocycloalkyl, —(CH₂)ₙ₁-aryl, —(CH₂)ₙ₁-heteroaryl, optionally substituted by halogen, lower alkyl, lower haloalkyl, lower alkoxy, lower haloalkoxy, lower hydroxyalkyl, hydroxy, C(═O)—R^{1b}, (C═O)—OR^{1b}, C(═O)—NH—R^{1b}, C(═O)—NH—CH₂—R^{1b}, or cyano; and

n₁ is 0 or 1.

In embodiment (ii), R¹ and R² are independently H or R^{2b};

each R^{2b} is independently lower alkyl, lower alkoxy, aryl, heteroaryl, cycloalkyl, heterocycloalkyl, or heterocycloalkyl alkylene, optionally substituted with one or more R^{2c};

R^{2c} is R^{2d} or R^{2e};

each R^{2d} is independently halogen, cyano, oxo, or hydroxy;

each R^{2e} is independently —OR^{2g}, —N(R^{2g})₂, —C(═O)(R^{2g}), —C(═O)O(R^{2g}), —C(═O)N(R^{2g})₂, —N(R^{2g})C(═O)(R^{2g}), —S(═O)₂(R^{2g}), —S(O)₂N(R^{2g})₂, lower alkyl, lower alkoxy, lower haloalkyl, aryl, heteroaryl, heteroaryloxy, cycloalkyl, or heterocycloalkyl, optionally substituted with one or more R^{2f};

each R^{2f} is independently H, halogen, lower alkyl, lower alkoxy, oxo, or lower haloalkyl; and

each R^{2g} is independently H, lower alkyl, lower alkoxy, lower haloalkyl, or aryl.

In embodiment (iii) R¹ is H and

R² is

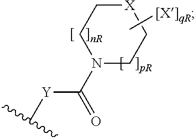

X is C(R^{3d})(R^{3e}), N(R^{3d}), S(═O)₂, or O;

each X′ is independently halogen, lower alkyl, cyano, hydroxy, C(═O)—OR^{3g}, C(═O)R^{3g}, lower haloalkyl, lower hydroxyalkyl, heteroaryl, spiroheterocycloalkyl, spirocycloalkyl, lower alkoxy, lower alkylamino, or lower dialkylamino;

or two adjacent X′ come together to form a ring which can be saturated or unsaturated;

Y is C(R^{3a})₂(C(R³ⁱ)₂)m_{R};

R^{3a} is independently H or R^{3b};

R^{3b} is lower alkyl, lower alkoxy, aryl, benzyl, heteroaryl, cycloalkyl, heterocycloalkyl, or cycloalkylalkyl, optionally substituted with one or more R^{3c};

R^{3c} is halogen, lower alkyl, lower haloalkyl, lower alkoxy, lower hydroxyalkyl, lower haloalkyl, oxo, hydroxy, or cyano;

each R^{3d} is independently H or R^{3f};

R^{3e} is H, hydroxy, halogen or lower alkyl;

or R^{3d} and R^{3e} come together to form a spirocyclic ring system, wherein the spirocyclic ring system can optionally include one or more heteroatoms or heteroatom containing moieties such as C═O and wherein the spirocyclic ring system can be optionally substituted with one or more R^{3h};

or X′ and R^{3d} come together to form a bicyclic ring system, optionally substituted with one or more R^{3h};

each R^{3f} is independently lower alkyl, lower haloalkyl, halogen, lower alkoxy, lower hydroxyalkyl, cycloalkyl, heterocycloalkyl, aryl, heteroaryl, lower alkylene-cycloalkyl, lower alkylene-heterocycloalkyl, lower alkylene-aryl, lower alkylene-heteroaryl, cyano, cyano lower alkyl, hydroxy, C(═O)—OR^{3g}, C(═O)R^{3g} or S(═O)₂R^{3g};

each R^{3g} is independently H, OR³ⁱ, aryl, heteroaryl, lower alkyl, cycloalkyl or heterocycloalkyl;

R^{3h} is halogen, lower alkyl, lower alkoxy, hydroxy, hydroxy lower alkyl, lower haloalkyl, lower hydroxyalkylcyano, cycloalkyl, heterocycloalkyl, aryl, heteroaryl, lower alkylene-cycloalkyl, lower alkylene-heterocycloalkyl, lower alkylene-aryl, lower alkylene-heteroaryl, —C(O)O—R^{3g} or —S(O)₂CH₃;

each R³ⁱ is independently H, lower alkyl, or lower haloalkyl.

m_{R} is 0 or 1; preferably 1.

n_{R} is 0 or 1.

p_{R} is 0 or 1; preferably 1.

q_{R} is 0, 1, 2, 3, or 4; preferably 0, 1 or 2, more preferably 0 or 1.

In embodiment (iv), R¹ is H or OH;

R² is aryl, heterocycloalkyl, heteroaryl or cycloalkyl, each optionally substituted with one or more R^{4a};

each R^{4a} is independently hydroxy, halo, oxo, lower alkyl, lower alkenyl, lower alkynyl, lower haloalkyl, lower alkoxy, lower haloalkoxy, lower hydroxyalkyl, amino, lower alkylamino, lower dialkylamino, cyano, lower cyanoalkyl, cycloalkyl, heterocycloalkyl, C(═O)R^{4b}, or S(═O)₂R^{4b}; and

each R^{4b} is independently OH, cycloalkyl or lower alkyl.

In embodiment (v), R¹ is H;

R² is lower alkoxy or

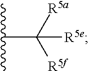

or R¹ and R² together form heterocycloalkyl, optionally substituted with halogen or cyano;

R^{5a} is H, cyano, lower alkyl, R^{5b}, R^{5q} or

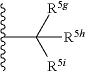

R^{5b} is cycloalkyl, heterocycloalkyl, heteroaryl, or aryl, wherein each is optionally substituted with one or more R^{5c};

each R^{5c} is independently halo, hydroxy, cyano, lower alkyl, lower haloalkyl, lower alkoxy, lower hydroxyalkyl, cycloalkyl, C(═O)R^{5d}, or S(═O)₂R^{5d};

each R^{5d} is independently OH or lower alkyl;

R^{5e} is H, hydroxy lower alkyl, lower haloalkyl, or lower alkyl;

R^{5f} is H, hydroxy, cyano, cyano lower alkyl, —C(═O)NH₂, —C(═O)OH, —C(═O)OC(CH₃)₃, R^{5r}, R^{5s} or R^{5k};

R^{5g} and R^{5h} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, C(═O)R^{5d}, S(═O)₂R^{5d} or CH₂S(═O)₂R^{5d}; R⁵ⁱ is aryl, cycloalkyl, heterocycloalkyl, or heteroaryl, optionally substituted with one or more R^{5j};

each R^{5j} is independently hydroxy, halo, lower alkyl, lower hydroxyalkyl, lower halo alkyl, or lower alkoxy;

each R^{5k} is independently lower alkyl, hydroxy lower alkyl, lower alkoxy, lower haloalkyl, lower haloalkoxy, aryl lower alkyl, cycloalkyl or cycloalkyl lower alkyl, each optionally substituted with one or more R^{5m};

each R^{5m} is independently lower alkyl, halo, hydroxy, lower alkoxy, lower haloalkyl, lower hydroxy alkyl, oxo, amino, cyano, cyano lower alkyl, S(═O)₂R⁵ⁿ, C(═O)R⁵ⁿ, cycloalkyl, heterocycloalkyl, heteroaryl, lower alkyl sulfonylamino, lower alkyl sulfonyl, halo lower alkoxy, cycloalkyl, —C(═O)OCH₃ or heterocycloalkenyl;

each R⁵ⁿ is independently H, hydroxy or lower alkyl;

each R^{5p} is independently hydroxy, amino, oxo, lower alkyl, —C(═O)NH₂, cyano, lower haloalkyl, benzyl, cyano lower alkyl, or —NHC(═O)OC(CH₃)₃;

R^{5q} is lower alkoxyl, hydroxy lower alkyl, or lower haloalkyl;

or R^{5q} and R^{5e} together form heterocycloalkyl, cycloalkyl, indan-1-yl, aryl, or heteroaryl, optionally substituted with one or more R^{5p};

R^{5′} is aryl, heteroaryl, heterocycloalkyl, heterocycloalkyl lower alkyl, heteroaryl lower alkyl, aryl lower alkoxy, optionally substituted with one or more R^{5m};

R^{5s} is —C(═O)R^{5t} or —CH₂C(═O)R^{5t};

R^{5t} is heterocycloalkyl, optionally substituted with one or more R^{5u}; and

each R^{5u} is independently cyano, halo, lower alkyl, or lower alkyl sulfonyl.

In a more preferred embodiment (vi), R¹ is H;

R² is

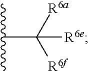

R^{6a} is H, cyano, lower alkyl, R^{6b} or

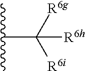

R^{6b} is cycloalkyl, heterocycloalkyl, heteroaryl, or aryl, wherein each is optionally substituted with one or more R^{6c};

each R^{6c} is independently halo, hydroxy, cyano, lower alkyl, lower haloalkyl, lower alkoxy, lower hydroxyalkyl, cycloalkyl, C(═O)R^{6d}, or S(═O)₂R^{6d};

each R^{6d} is independently OH or lower alkyl;

R^{6e} is H, hydroxy lower alkyl, lower haloalkyl, or lower alkyl;

R^{6f} is H, hydroxy, cyano, cyano lower alkyl, or R^{6k};

R^{6g} and R^{6h} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, C(═O)R^{6d}, S(═O)₂R^{6d} or CH₂S(═O)₂R^{6d};

R⁶ⁱ is aryl, cycloalkyl, heterocycloalkyl, or heteroaryl, optionally substituted with one or more R^{6j};

each R^{6j} is independently hydroxy, halo, lower alkyl, lower hydroxyalkyl, lower halo alkyl, or lower alkoxy;

each R^{6k} is independently lower alkyl, hydroxy lower alkyl, lower alkoxy, lower haloalkyl, lower haloalkoxy, aryl lower alkyl, cycloalkyl or cycloalkyl lower alkyl, each optionally substituted with one or more R^{6m};

each R^{6m} is independently lower alkyl, halo, hydroxy, lower alkoxy, lower haloalkyl, lower hydroxy alkyl, oxo, amino, cyano, cyano lower alkyl, S(═O)₂R⁶ⁿ, C(═O)R⁶ⁿ, cycloalkyl, heterocycloalkyl, heteroaryl, or heterocycloalkenyl; and

each R⁶ⁿ is independently H, hydroxy or lower alkyl.

In a more preferred embodiment (vii), R¹ is H;

R² is lower alkoxy or

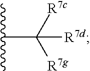

or R¹ and R² together form heterocycloalkyl, optionally substituted with halogen or cyano;

R^{7c} is H or R^{7f};

R^{7d} is H or lower alkyl;

each R^{7e} is independently hydroxy, amino, oxo, lower alkyl, —C(═O)NH₂, cyano, lower haloalkyl, benzyl, cyano lower alkyl, or —NHC(═O)OC(CH₃)₃;

R^{7f} is lower alkyl, cycloalkyl, lower alkoxyl, hydroxy lower alkyl, or lower haloalkyl;

or R^{7f} and R^{7d} together form heterocycloalkyl, cycloalkyl, indan-1-yl, aryl, or heteroaryl, optionally substituted with one or more R^{7e};

R^{7g} is H, hydroxy, cyano, —C(═O)NH₂, —C(═O)OH, —C(═O)OC(CH₃)₃, R^{7h}, or R^{7j};

R^{7h} is lower alkyl, aryl, aryl lower alkyl, cycloalkyl, heteroaryl, heterocycloalkyl, heterocycloalkyl lower alkyl, heteroaryl lower alkyl, aryl lower alkoxy, lower alkoxy, optionally substituted with one or more R⁷ⁱ;

each R⁷ⁱ is independently hydroxy, cyano, amino, lower alkyl sulfonylamino, lower alkoxy, halo, lower alkyl, cyano lower alkyl, lower haloalkyl, lower alkyl sulfonyl, oxo, halo lower alkoxy, cycloalkyl, —C(═O)OCH₃;

R^{7j} is —C(═O)R^{7k} or —CH₂C(═O)R^{7k};

R^{7k} is heterocycloalkyl, optionally substituted with one or more R^{7m}; and

each R^{7m} is independently cyano, halo, lower alkyl, or lower alkyl sulfonyl.

R¹ and R² are preferably as defined in option (iii) or (ii).

In an even more preferred embodiment,

R¹ is H;

R² is —CHR^{1a}—C(O)—NR^{1e}R^{1g};

R^{1a} is cycloalkyl (preferably cyclopropyl), H, or lower alkyl;

R^{1d} is cyano, —(CH₂)ₙ₁—R**, C(O)—(CH₂)ₙ₁—R** or C(O)—NH—(CH₂)ₙ₁—R**, wherein R** is optionally substituted with one or more of halogen, lower haloalkyl, (C═O)—OR*, lower alkyl, lower alkoxy, lower haloalkoxy, or cyano;

R^{1e} is H, cycloalkyl, aryl or lower alkyl, wherein cycloalkyl, aryl or lower alkyl can be optionally substituted with one or more R^{1d}; more preferably R^{1e} is cycloalkyl, aryl or lower alkyl, wherein cycloalkyl, aryl or lower alkyl can be optionally substituted with one or more R^{1d}.

R^{1g} is H;

R* is H or lower alkyl;

R** is cycloalkyl, aryl, heterocycloalkyl or heteroaryl; and

n₁ is 0 or 1.

In an even more preferred embodiment,

R¹ is H;

R² is lower alkyl, heterocycloalkyl, aryl, heterocycloalkyl or cycloalkyl, wherein lower alkyl, cycloalkyl, aryl, heterocycloalkyl or cycloalkyl can be optionally substituted with one or more R^{2c}; more preferably R² is lower alkyl, heterocycloalkyl or cycloalkyl, optionally substituted with one or more R^{2c};

R^{2c} is cycloalkyl, heterocycloalkyl, heteroaryl, aryl, OR*, halogen, cyano, COOR* or —S(O)₂—R*, wherein cycloalkyl, heterocycloalkyl, heteroaryl and aryl can be optionally substituted by lower alkyl, lower alkoxy, or lower haloalkyl; more preferably R^{2c} is heteroaryl, aryl, cyano, COOR* or —S(O)₂—R*, wherein heteroaryl and aryl can be optionally substituted by lower alkyl, lower alkoxy, or lower haloalkyl; and and

R* is H or lower alkyl.

In an even more preferred embodiment,

R¹ is H;

R² is

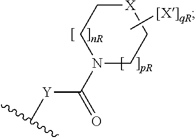

X′ is halogen, hydroxy, lower hydroxyalkyl, C(O)OR^{3g} or C(O)R^{3g};

or adjacent X′ come together to form a ring (such as an aromatic ring) which can be saturated or unsaturated;

Y is CH(R^{3b});

n_{R} is 0 or 1;

p_{R} is 0 or 1;

q_{R} is 0 or 1;

R^{3b} is H, cycloalkyl or lower alkyl;

R^{3g} is OR*, cycloalkyl, aryl, heterocycloalkyl, or heteroaryl; more preferably R^{3g} is heterocycloalkyl;

R* is H or lower alkyl;

X is CF₂, CH₂, O, or N(R^{3d}) in which R^{3d} is lower alkylene-aryl, heterocycloalkyl; or X is C(R^{3d})(R^{3e}) in which R^{3d} and R^{3e} come together to form a (e.g., four to six-membered) spirocyclic ring system which can optionally include one or more heteroatoms (e.g., N, O or S) or heteroatom containing moieties and wherein the spirocyclic ring system can be optionally substituted with one or more R^{3h} such as benzyl or —C(O)O—R*.

The compounds of the present invention can be administered to a patient in the form of a pharmaceutical composition which can optionally comprise one or more pharmaceutically acceptable excipient(s) and/or carrier(s).

The compounds of the present invention can be administered by various well known routes, including oral, rectal, intragastrical, intracranial and parenteral administration, e.g. intravenous, intramuscular, intranasal, intradermal, subcutaneous, and similar administration routes. Oral, intranasal and parenteral administration are particularly preferred. Depending on the route of administration different pharmaceutical formulations are required and some of those may require that protective coatings are applied to the drug formulation to prevent degradation of a compound of the invention in, for example, the digestive tract.

Thus, preferably, a compound of the invention is formulated as a syrup, an infusion or injection solution, a spray, a tablet, a capsule, a capslet, lozenge, a liposome, a suppository, a plaster, a band-aid, a retard capsule, a powder, or a slow release formulation. Preferably the diluent is water, a buffer, a buffered salt solution or a salt solution and the carrier preferably is selected from the group consisting of cocoa butter and vitebesole.

Particular preferred pharmaceutical forms for the administration of a compound of the invention are forms suitable for injectable use and include sterile aqueous solutions or dispersions and sterile powders for the extemporaneous preparation of sterile injectable solutions or dispersion. In all cases the final solution or dispersion form must be sterile and fluid. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. A compound of the invention can also be formulated into liposomes, in particular for parenteral administration. Liposomes provide the advantage of increased half life in the circulation, if compared to the free drug and a prolonged more even release of the enclosed drug.

Sterilization of infusion or injection solutions can be accomplished by any number of art recognized techniques including but not limited to addition of preservatives like anti-bacterial or anti-fungal agents, e.g. parabene, chlorobutanol, phenol, sorbic acid or thimersal. Further, isotonic agents, such as sugars or salts, in particular sodium chloride may be incorporated in infusion or injection solutions.

Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions are vacuum-dried or freeze-dried as necessary. Preferred diluents of the present invention are water, physiological acceptable buffers, physiological acceptable buffer salt solutions or salt solutions. Preferred carriers are cocoa butter and vitebesole. Excipients which can be used with the various pharmaceutical forms of a compound of the invention can be chosen from the following non-limiting list:

a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;

b) lubricants such as magnesium stearate, talc, calcium stearate, zinc stearate, stearic acid, hydrogenated vegetable oil, leucine, glycerids and sodium stearyl fumarates,

c) disintegrants such as starches, croscaramellose, sodium methyl cellulose, agar, bentonite, alginic acid, carboxymethyl cellulose, polyvinyl pyrrolidone and the like.

In one embodiment the formulation is for oral administration and the formulation comprises one or more or all of the following ingredients: pregelatinized starch, talc, povidone K 30, croscarmellose sodium, sodium stearyl fumarate, gelatin, titanium dioxide, sorbitol, monosodium citrate, xanthan gum, titanium dioxide, flavoring, sodium benzoate and saccharin sodium.

If a compound of the invention is administered intranasally in a preferred embodiment, it may be administered in the form of a dry powder inhaler or an aerosol spray from a pressurized container, pump, spray or nebulizer with the use of a suitable propellant, e.g., dichlorodifluoromethane, trichlorofluoromethane, dichlorotetrafluoroethane, a hydrofluoro-alkane such as 1,1,1,2-tetrafluoroethane (HFA 134A™) or 1,1,1,2,3,3,3-heptafluoropropane (HFA 227EA™), carbon dioxide, or another suitable gas. The pressurized container, pump, spray or nebulizer may contain a solution or suspension of the compound of the invention, e.g., using a mixture of ethanol and the propellant as the solvent, which may additionally contain a lubricant, e.g., sorbitan trioleate.

Other suitable excipients can be found in the Handbook of Pharmaceutical Excipients, published by the American Pharmaceutical Association, which is herein incorporated by reference.

It is to be understood that depending on the severity of the disorder and the particular type which is treatable with one of the compounds of the invention, as well as on the respective patient to be treated, e.g. the general health status of the patient, etc., different doses of the respective compound are required to elicit a therapeutic or prophylactic effect. The determination of the appropriate dose lies within the discretion of the attending physician. It is contemplated that the dosage of a compound of the invention in the therapeutic or prophylactic use of the invention should be in the range of about 0.1 mg to about 1 g of the active ingredient (i.e. compound of the invention) per kg body weight. However, in a preferred use of the present invention a compound of the invention is administered to a subject in need thereof in an amount ranging from 1.0 to 500 mg/kg body weight, preferably ranging from 1 to 200 mg/kg body weight. The duration of therapy with a compound of the invention will vary, depending on the severity of the disease being treated and the condition and idiosyncratic response of each individual patient. In one preferred embodiment of a prophylactic or therapeutic use, between 100 mg to 200 mg of the compound is orally administered to an adult per day, depending on the severity of the disease and/or the degree of exposure to disease carriers.

As is known in the art, the pharmaceutically effective amount of a given composition will also depend on the administration route. In general the required amount will be higher, if the administration is through the gastrointestinal tract, e.g., by suppository, rectal, or by an intragastric probe, and lower if the route of administration is parenteral, e.g., intravenous. Typically, a compound of the invention will be administered in ranges of 50 mg to 1 g/kg body weight, preferably 100 mg to 500 mg/kg body weight, if rectal or intragastric administration is used and in ranges of 10 to 100 mg/kg body weight, if parenteral administration is used.

If a person is known to be at risk of developing a disease treatable with a compound of the invention, prophylactic administration of the biologically active blood serum or the pharmaceutical composition according to the invention may be possible. In these cases the respective compound of the invention is preferably administered in above outlined preferred and particular preferred doses on a daily basis. Preferably, from 0.1 mg to 1 g/kg body weight once a day, preferably 10 to 200 mg/kg body weight. This administration can be continued until the risk of developing the respective disorder has lessened. In most instances, however, a compound of the invention will be administered once a disease/disorder has been diagnosed. In these cases it is preferred that a first dose of a compound of the invention is administered one, two, three or four times daily.

The compounds of the present invention are particularly useful for treating, ameliorating, or preventing influenza. Within the present invention, the term “influenza” includes influenza A, B, C, isavirus and thogotovirus and also covers bird flu and swine flu. The subject to be treated is not particularly restricted and can be any vertebrate, such as birds and mammals (including humans).

Without wishing to be bound by theory it is assumed that the compounds of the present invention are capable of inhibiting binding of host mRNA cap structures to the cap-binding domain (CBD), particularly of the influenza virus. More specifically it is assumed that they directly interfere with the CBD of the influenza PB2 protein. However, delivery of a compound into a cell may represent a problem depending on, e.g., the solubility of the compound or its capabilities to cross the cell membrane. The present invention not only shows that the claimed compounds have in vitro polymerase inhibitory activity but also in vivo antiviral activity.

A possible measure of the in vivo antiviral activity of the compounds having the formula I or (I) is the CPE assay disclosed herein. Preferably the compounds exhibit a % reduction of at least about 30% at 50 μM. In this connection, the reduction in the virus-mediated cytopathic effect (CPE) upon treatment with the compounds was calculated as follows: The cell viability of infected-treated and uninfected-treated cells was determined using an ATP-based cell viability assay (Promega). The response in relative luminescent units (RLU) of infected-untreated samples was subtracted from the response (RLU) of the infected-treated samples and then normalized to the viability of the corresponding uninfected sample resulting in % CPE reduction. Preferably the compounds exhibit an IC₅₀ of at least about 45 μM, more preferably at least about 10 μM, in the CPE assay. The half maximal inhibitory concentration (IC₅₀) is a measure of the effectiveness of a compound in inhibiting biological or biochemical function and was calculated from the RLU response in a given concentration series ranging from maximum 100 μM to at least 100 nM.

The compounds having the general formula (I) can be used in combination with one or more other medicaments. The type of the other medicaments is not particularly limited and will depend on the disorder to be treated. Preferably the other medicament will be a further medicament which is useful in treating, ameliorating or preventing a viral disease, more preferably a further medicament which is useful in treating, ameliorating or preventing influenza.

The following combinations of medicaments are envisaged as being particularly suitable:
(i) The combination of endonuclease and cap binding inhibitors (particularly targeting influenza). The endonuclease inhibitors are not particularly limited and can be any endonuclease inhibitor, particularly any viral endonuclease inhibitor.
   Widespread resistance to both classes of licensed influenza antivirals (M2 ion channel inhibitors (adamantanes) and neuraminidase inhibitors (Oseltamivir)) occurs in both pandemic and seasonal viruses, rendering these drugs to be of marginal utility in the treatment modality. For M2 ion channel inhibitors, the frequency of viral resistance has been increasing since 2003 and for seasonal influenza A/H3N2, adamantanes are now regarded as ineffective. Virtually all 2009 H1N1 and seasonal H3N2 strains are resistant to the adamantanes (rimantadine and amantadine), and the majority of seasonal H1N1 strains are resistant to oseltamivir, the most widely prescribed neuraminidase inhibitor (NAI). For oseltamivir the WHO reported on significant emergence of influenza A/H1N1 resistance starting in the influenza season 2007/2008; and for the second and third quarters of 2008 in the southern hemisphere. Even more serious numbers were published for the fourth quarter of 2008 (northern hemisphere) where 95% of all tested isolates revealed no Oseltamivir-susceptibility. Considering the fact that now most national governments have been stockpiling Oseltamivir as part of their influenza pandemic preparedness plan, it is obvious that the demand for new, effective drugs is growing significantly. To address the need for more effective therapy, preliminary studies using double or even triple combinations of antiviral drugs with different mechanisms of action have been undertaken. Adamantanes and neuraminidase inhibitors in combination were analysed in vitro and in vivo and found to act highly synergistically. However, it is known that for both types of antivirals resistant viruses emerge rather rapidly and this issue is not tackled by combining these established antiviral drugs.
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. These two targets are located within distinct subunits of the polymerase complex and thus represent unique drug targets. Due to the fact that both functions are required for the so-called “cap-snatching” mechanism mandatory for viral transcription, concurrent inhibition of both functions is expected to act highly synergistically. This highly efficient drug combination would result in lower substance concentrations and hence improved dose-response-relationships and better side effect profiles.
   Both of these active sites are composed of identical residues in all influenza A strains (e.g., avian and human) and hence this high degree of sequence conservation underpins the perception that these targets are not likely to trigger rapid resistant virus generation. Thus, endonuclease and cap-binding inhibitors individually and in combination are ideal drug candidates to combat both seasonal and pandemic influenza, irrespectively of the virus strain.
   The combination of an endonuclease inhibitor and a cap-binding inhibitor or a dual specific polymerase inhibitor targeting both the endonuclease active site and the cap-binding domain would be effective against virus strains resistant against adamantanes and neuraminidase inhibitors and moreover combine the advantage of low susceptibility to resistance generation with activity against a broad range of virus strains.
(ii) The combination of inhibitors of different antiviral targets (particularly targeting influenza) focusing on the combination with (preferably influenza) polymerase inhibitors as dual or multiple combination therapy. Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of a different antiviral target is expected to act highly synergistically. This is based on the fact that these different types of antiviral drugs exhibit completely different mechanisms of action and pharmacokinetics properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the first group of polymerase inhibitors is combined with at least one compound selected from the second group of polymerase inhibitors.
   The first group of polymerase inhibitors which can be used in this type of combination therapy includes, but is not limited to, the compounds having the general formula (I) described below, the compounds having the general formula ((I)) described above and/or the compounds disclosed in WO2011/000566.
   The second group of polymerase inhibitors which can be used in this type of combination therapy includes, but is not limited to, compounds disclosed in WO 2010/110231, WO 2010/110409, WO 2006/030807 and U.S. Pat. No. 5,475,109 as well as flutimide and analogues, favipiravir and analogues, epigallocatechin gallate and analogues, as well as nucleoside analogs such as ribavirine.
(iii) The combination of polymerase inhibitors with neuramidase inhibitors
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of a different extracellular antiviral target, especially the (e.g., viral) neuraminidase is expected to act highly synergistically. This is based on the fact that these different types of antiviral drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the above mentioned first group of polymerase inhibitors is combined with at least one neuramidase inhibitor.
   The neuraminidase inhibitor (particularly influenza neuramidase inhibitor) is not specifically limited. Examples include zanamivir, oseltamivir, peramivir, KDN DANA, FANA, and cyclopentane derivatives.
(iv) The combination of polymerase inhibitors with M2 channel inhibitors
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of a different extracellular and cytoplasmic antiviral target, especially the viral M2 ion channel, is expected to act highly synergistically. This is based on the fact that these different types of antiviral drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the above mentioned first group of polymerase inhibitors is combined with at least one M2 channel inhibitor.
   The M2 channel inhibitor (particularly influenza M2 channel inhibitor) is not specifically limited. Examples include amantadine and rimantadine.
(v) The combination of polymerase inhibitors with alpha glucosidase inhibitors
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target, with an inhibitor of a different extracellular target, especially alpha glucosidase, is expected to act highly synergistically. This is based on the fact that these different types of antiviral drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the above mentioned first group of polymerase inhibitors is combined with at least one alpha glucosidase inhibitor.
   The alpha glucosidase inhibitor (particularly influenza alpha glucosidase inhibitor) is not specifically limited. Examples include the compounds described in Chang et al., Antiviral Research 2011, 89, 26-34.
(vi) The combination of polymerase inhibitors with ligands of other influenza targets
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of different extracellular, cytoplasmic or nucleic antiviral targets is expected to act highly synergistically. This is based on the fact that these different types of antiviral drugs exhibit completely different mechanisms of action and pharmacokinetic properties which act advantageously and synergistically on the antiviral efficacy of the combination.

This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
Typically at least one compound selected from the above mentioned first group of polymerase inhibitors is combined with at least one ligand of another influenza target.
The ligand of another influenza target is not specifically limited. Examples include compounds acting on the sialidase fusion protein, e.g. Fludase (DAS181), siRNAs and phosphorothioate oligonucleotides, signal transduction inhibitors (ErbB tyrosine kinase, Abl kinase family, MAP kinases, PKCa-mediated activation of ERK signaling as well as interferon (inducers).
(vii) The combination of (preferably influenza) polymerase inhibitors with a compound used as an adjuvance to minimize the symptoms of the disease (antibiotics, anti-inflammatory agents like COX inhibitors (e.g., COX-1/COX-2 inhibitors, selective COX-2 inhibitors), lipoxygenase inhibitors, EP ligands (particularly EP4 ligands), bradykinin ligands, and/or cannabinoid ligands (e.g., CB2 agonists). Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with a compound used as an adjuvance to minimize the symptoms of the disease address the causative and symptomatic pathological consequences of viral infection. This combination is expected to act synergistically because these different types of drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.

The present invention not only shows that the compounds have in vitro polymerase inhibitory activity but also in vivo antiviral activity.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention as indicated by the appended claims in any way.

### Examples

### Fp Assay

Surface Plasmon Resonance Measurements (SPR)

SPR was performed on a Biacore X100 system equipped with CM7 sensor chips (GE Healthcare). The expression construct for PB2 cap binding domain (PB2-CBD) (residues 318-483) of the avian influenza strain A/duck/Shantou/4610/2003(H5N1) was synthesized by Geneart AG. Purified protein was kindly provided by Stephen Cusack and his co-workers (EMBL Grenoble, Guilligay et al., 2008). The protein concentration was determined by OD₂₈₀ measurement using the extinction coefficient of 6990 M¹·cm⁻¹ at 280 nm. PB2-CBD was immobilized on the sensor surface by amine coupling according to the manufacturer's protocol using a protein concentration of 30 μg ml⁻¹ and 5 mM m7GTP (Sigma-Aldrich) in 10 mM phosphate buffer pH 6.5 and HBS-EP buffer (GE Healthcare). Compound testing was performed in running buffer (10 mM TRIS, 3 mM EDTA, 150 mM NaCl, 0.005% (v/v) Surfactant p20 (GE Healthcare/Biacore), 1 mM DTT) at a final DMSO concentration of 0.5% (v/v) DMSO and a flow rate of 10 μl min⁻¹. Sensorgrams were processed using double referencing and solvent correction for DMSO bulk effects. Affinity constants (K_{d} values) were determined using a linear curve fit model of Biacore X100 Evaluation Software.

Cap Fluorescence-Polarization Ligand Displacement (CapFP-LD) Assay

The expression construct for PB2 cap binding domain (PB2-CBD) (residues 318-483) of the avian influenza strain A/duck/Shantou/4610/2003(H5N1) was synthesized by Geneart AG. Purified protein was kindly provided by Stephen Cusack and his co-workers (EMBL Grenoble; Guilligay et al., 2008). PB2-CBD concentration was determined by OD₂₈₀ measurement using the extinction coefficient of 6990 M⁻¹·cm⁻¹ at 280 nm, m⁷GTP-5FAM (Jena Bioscience) was used as fluorescent tracer. The concentrations of tracer and receptor were chosen according to their K_{d} value of 0.42 μM determined in assay buffer (10 mM HEPES pH 7.4, 100 mM NaAc, 10 mM Mg(Ac)₂, 0.005% (v/v) protein-grade TWEEN 20) (Nikolovska-Coleska et al., 2004). A series of 2-fold dilutions of compound were prepared, transferred to 384-well plates (Corning #3676) at a final DMSO concentration of 10% (v/v). The tracer/protein mixture was added to a final concentration of 2 μM and 1.2 μM respectively. The plates were sealed and incubated shaking for 30 min before FP was measured. The data was analysed using GraphPad Prism to determine IC₅₀ values and 95% confidence intervals using a 4-parameter logistic equation. Positive and negative controls were included to define top and bottom for curve fitting.

Cytopathic Effect (CPE) Assay

The influenza A virus (IAV) was obtained from American Tissue Culture Collection (A/Aichi/2/68 (H3N2); VR-547). Virus stocks were prepared by propagation of virus on Mardin-Darby canine kidney (MDCK; ATCC CCL-34) cells and infectious titres of virus stocks were determined by the 50% tissue culture infective dose (TCID₅₀) analysis as described in Reed, L. J., and H. Muench., Am. J. Hyg. 1938, 27:493-497.

MDCK cells were seeded in 96-well plates at 2×10⁴ cells/well using DMEM/Ham's F-12 (1:1) medium containing 10% foetal bovine serum (FBS), 2 mM L-glutamine and 1% antibiotics (all from PAA). Until infection the cells were incubated for 5 hrs at 37° C., 5.0% CO₂ to form a ˜80% confluent monolayer on the bottom of the well. Each test compound was dissolved in DMSO and generally tested at 25 μM and 250 μM. In those cases where the compounds were not soluble at that concentration they were tested at the highest soluble concentration. The compounds were diluted in infection medium (DMEM/Ham's F-12 (1:1) containing 5 μg/ml trypsin, and 1% antibiotics) for a final plate well DMSO concentration of 1%. The virus stock was diluted in infection medium (DMEM/Ham's F-12 (1:1) containing 5 μg/ml Trypsin, 1% DMSO, and 1% antibiotics) to a theoretical multiplicity of infection (MOI) of 0.05.

After removal of the culture medium and one washing step with PBS, virus and compound were added together to the cells. In the wells used for cytotoxicity determination (i.e. in the absence of viral infection), no virus suspension was added. Instead, infection medium was added. Each treatment was conducted in two replicates. After incubation at 37° C., 5% CO₂ for 48 hrs, each well was observed microscopically for apparent cytotoxicity, precipitate formation, or other notable abnormalities. Then, cell viability was determined using CellTiter-Glo luminescent cell viability assay (Promega). The supernatant was removed carefully and 65 μl of the reconstituted reagent were added to each well and incubated with gentle shaking for 15 min at room temperature. Then, 60 μl of the solution was transferred to an opaque plate and luminescence (RLU) was measured using Synergy HT plate reader (Biotek).

Relative cell viability values of uninfected-treated versus uninfected-untreated cells were used to evaluate cytotoxicity of the compounds. Substances with a relative viability below 80% at the tested concentration were regarded as cytotoxic and retested at lower concentrations.

Reduction in the virus-mediated cytopathic effect (CPE) upon treatment with the compounds was calculated as follows: The response (RLU) of infected-untreated samples was subtracted from the response (RLU) of the infected-treated samples and then normalized to the viability of the corresponding uninfected sample resulting in % CPE reduction. The half maximal inhibitory concentration (IC₅₀) is a measure of the effectiveness of a compound in inhibiting biological or biochemical function and was calculated from the RLU response in a given concentration series ranging from a maximum 100 μM to at least 100 nM.

The compounds were synthesized following the procedures which are set out in WO 2009/106441, WO2009/106442, WO 2009/106444; WO 2011/117145, WO 2011/117160, WO 2011/144584, and WO 2011/144585.

The activity of the compounds was investigated using the Fp and CPE assays. The results are given in the following table.

| Formula | Fp | CPE |
|---|---|---|
| 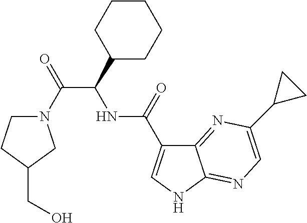 [SMILES: O=C(N[C@@H](C(=O)N1CCC(CO)C1)C1CCCCC1)c1c[nH]c2ncc(C3CC3)nc12] | | −10.1% @ 50 μM |
| 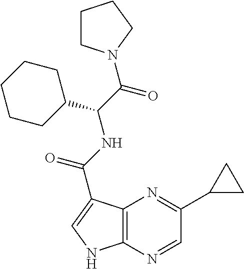 [SMILES: O=C(N[C@@H](C(=O)N1CCCC1)C1CCCCC1)c1c[nH]c2ncc(C3CC3)nc12] | | |
| 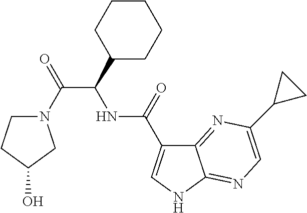 [SMILES: O=C(N[C@@H](C(=O)N1CC[C@@H](O)C1)C1CCCCC1)c1c[nH]c2ncc(C3CC3)nc12] | | −14.8% @ 50 μM |
| 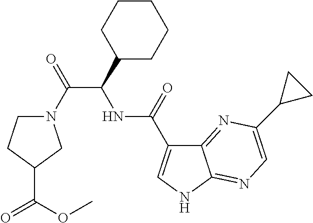 [SMILES: COC(=O)C1CCN(C(=O)[C@H](NC(=O)c2c[nH]c3ncc(C4CC4)nc23)C2CCCCC2)C1] | | −8.1% @ 5 μM |
| 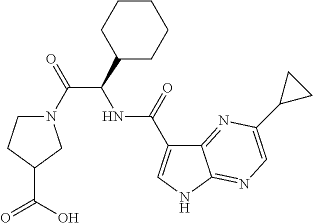 [SMILES: O=C(N[C@@H](C(=O)N1CCC(C(=O)O)C1)C1CCCCC1)c1c[nH]c2ncc(C3CC3)nc12] | | 11.6% @ 5 μM |
| 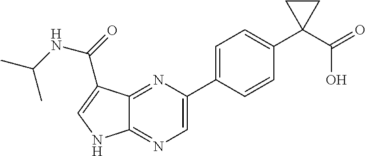 [SMILES: CC(C)NC(=O)c1c[nH]c2ncc(-c3ccc(C4(C(=O)O)CC4)cc3)nc12] | | −8.7% @ 25 μM |
| 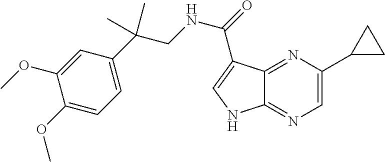 [SMILES: COc1ccc(C(C)(C)CNC(=O)c2c[nH]c3ncc(C4CC4)nc23)cc1OC] | | 15.8% @ 2.5 μM |
| 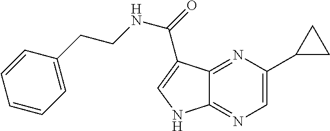 [SMILES: O=C(NCCc1ccccc1)c1c[nH]c2ncc(C3CC3)nc12] | | IC50 66.9 μM; CC50 > 100 μM |
| 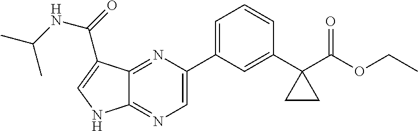 [SMILES: CCOC(=O)C1(c2cccc(-c3cnc4[nH]cc(C(=O)NC(C)C)c4n3)c2)CC1] | | −0.5% @ 5 μM |
| 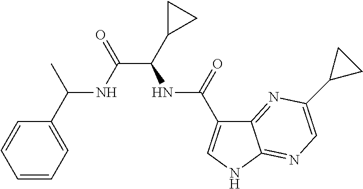 [SMILES: CC(NC(=O)[C@H](NC(=O)c1c[nH]c2ncc(C3CC3)nc12)C1CC1)c1ccccc1] | | 2.7% @ 5 μM |
| 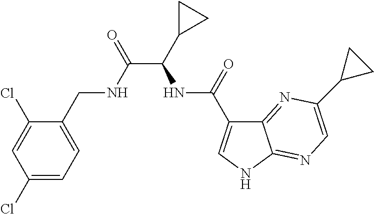 [SMILES: O=C(N[C@@H](C(=O)NCc1ccc(Cl)cc1Cl)C1CC1)c1c[nH]c2ncc(C3CC3)nc12] | | IC50 11.2 μM; CC50 > 12.5 μM |
| 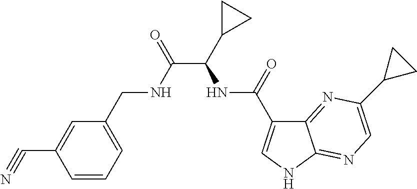 [SMILES: N#Cc1cccc(CNC(=O)[C@H](NC(=O)c2c[nH]c3ncc(C4CC4)nc23)C2CC2)c1] | | IC50 12.4 μM; CC50 > 50 μM |
| 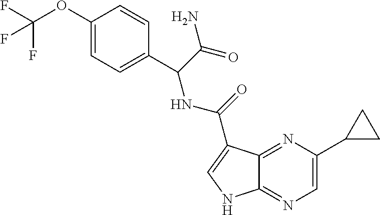 [SMILES: NC(=O)C(NC(=O)c1c[nH]c2ncc(C3CC3)nc12)c1ccc(OC(F)(F)F)cc1] | | −12.9% @ 5 μM |
| 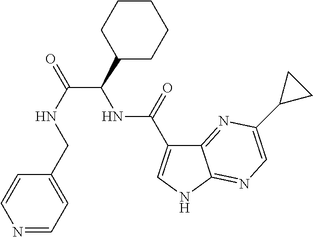 [SMILES: O=C(N[C@@H](C(=O)NCc1ccncc1)C1CCCCC1)c1c[nH]c2ncc(C3CC3)nc12] | | −8.3% @ 50 μM |
| 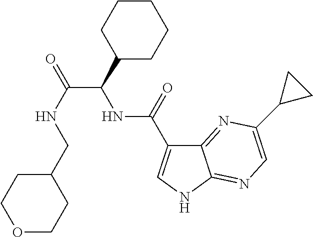 [SMILES: O=C(N[C@@H](C(=O)NCC1CCOCC1)C1CCCCC1)c1c[nH]c2ncc(C3CC3)nc12] | | 20.5% @ 5 μM |
| 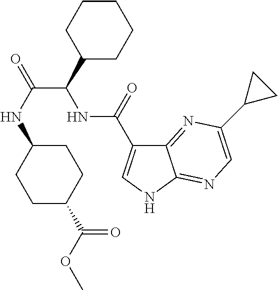 [SMILES: COC(=O)[C@H]1CC[C@H](NC(=O)[C@H](NC(=O)c2c[nH]c3ncc(C4CC4)nc23)C2CCCCC2)CC1] | | Active |
| 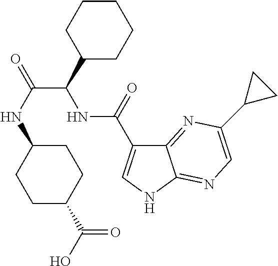 [SMILES: O=C(N[C@@H](C(=O)N[C@H]1CC[C@H](C(=O)O)CC1)C1CCCCC1)c1c[nH]c2ncc(C3CC3)nc12] | | 14.8% @ 50 μM |
| 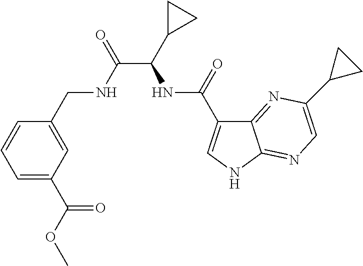 [SMILES: COC(=O)c1cccc(CNC(=O)[C@H](NC(=O)c2c[nH]c3ncc(C4CC4)nc23)C2CC2)c1] | | IC50 4.8 μM; CC50 63.9 μM |
| 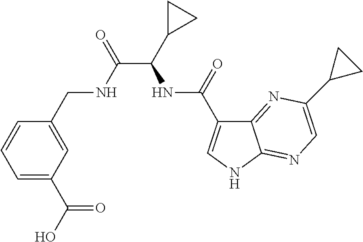 [SMILES: O=C(O)c1cccc(CNC(=O)[C@H](NC(=O)c2c[nH]c3ncc(C4CC4)nc23)C2CC2)c1] | | −0.2% @ 50 μM |
| 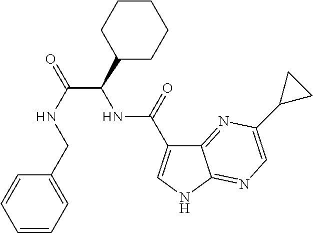 [SMILES: O=C(N[C@@H](C(=O)NCc1ccccc1)C1CCCCC1)c1c[nH]c2ncc(C3CC3)nc12] | | −2.2% @ 5 μM |
| 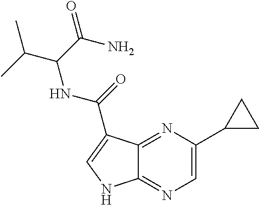 [SMILES: CC(C)C(NC(=O)c1c[nH]c2ncc(C3CC3)nc12)C(N)=O] | | −10.7% @ 5 μM |
| 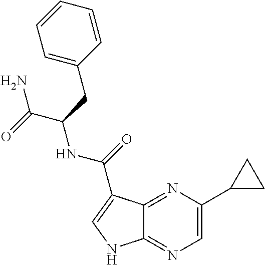 [SMILES: NC(=O)[C@@H](Cc1ccccc1)NC(=O)c1c[nH]c2ncc(C3CC3)nc12] | | |
| 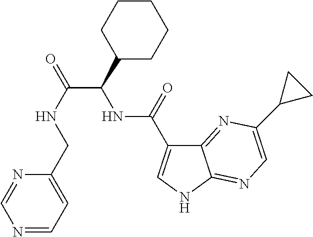 [SMILES: O=C(N[C@@H](C(=O)NCc1ccncn1)C1CCCCC1)c1c[nH]c2ncc(C3CC3)nc12] | | −2.2% @ 50 μM |
| 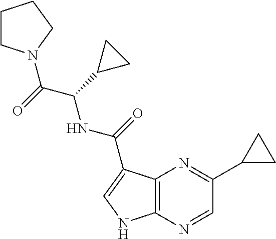 [SMILES: O=C(N[C@H](C(=O)N1CCCC1)C1CC1)c1c[nH]c2ncc(C3CC3)nc12] | Ki = 0.14 μM | IC50 > 25 μM |
| 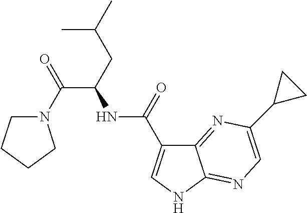 [SMILES: CC(C)C[C@@H](NC(=O)c1c[nH]c2ncc(C3CC3)nc12)C(=O)N1CCCC1] | Ki = 0.7 μM | IC50 > 100 μM |
| 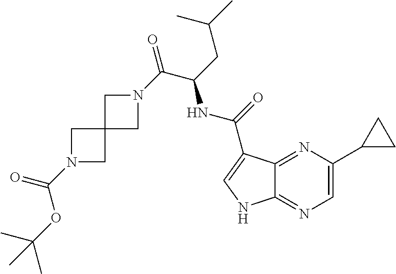 [SMILES: CC(C)C[C@@H](NC(=O)c1c[nH]c2ncc(C3CC3)nc12)C(=O)N1CC2(CN(C(=O)OC(C)(C)C)C2)C1] | Ki = 1.06 μM | IC50 > 100 μM |
| 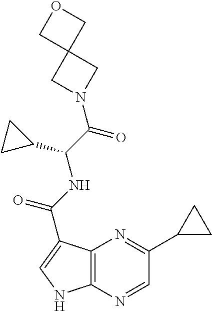 [SMILES: O=C(N[C@@H](C(=O)N1CC2(COC2)C1)C1CC1)c1c[nH]c2ncc(C3CC3)nc12] | Ki = 1.9 μM | IC50 > 100 μM |
| 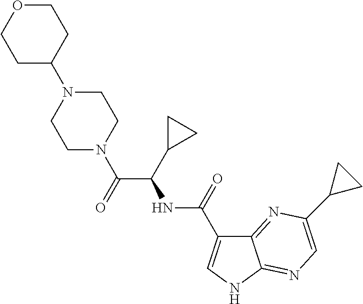 [SMILES: O=C(N[C@@H](C(=O)N1CCN(C2CCOCC2)CC1)C1CC1)c1c[nH]c2ncc(C3CC3)nc12] | Ki = 10.0 μM | IC50 > 100 μM |
| 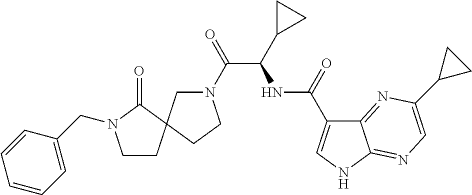 [SMILES: O=C(N[C@@H](C(=O)N1CCC2(CCN(Cc3ccccc3)C2=O)C1)C1CC1)c1c[nH]c2ncc(C3CC3)nc12] | Ki = 11.34 μM | IC50 27 μM |
| 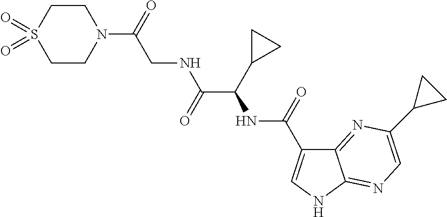 [SMILES: O=C(N[C@@H](C(=O)NCC(=O)N1CCS(=O)(=O)CC1)C1CC1)c1c[nH]c2ncc(C3CC3)nc12] | Ki = 11.9 μM | IC50 > 100 μM |
| 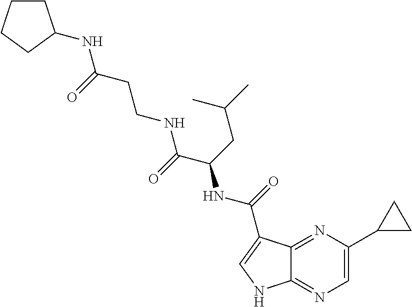 [SMILES: CC(C)C[C@@H](NC(=O)c1c[nH]c2ncc(C3CC3)nc12)C(=O)NCCC(=O)NC1CCCC1] | Ki = 12.20 μM | IC50 > 100 μM |
| 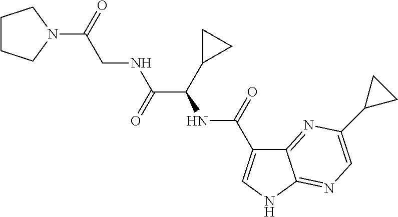 [SMILES: O=C(N[C@@H](C(=O)NCC(=O)N1CCCC1)C1CC1)c1c[nH]c2ncc(C3CC3)nc12] | Ki = 13.2 μM | IC50 > 100 μM |
| 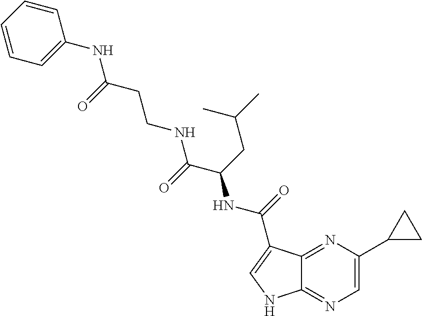 [SMILES: CC(C)C[C@@H](NC(=O)c1c[nH]c2ncc(C3CC3)nc12)C(=O)NCCC(=O)Nc1ccccc1] | Ki = 13.87 μM | IC50 > 100 μM |
| 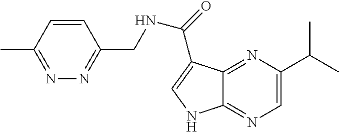 [SMILES: Cc1ccc(CNC(=O)c2c[nH]c3ncc(C(C)C)nc23)nn1] | Ki = 14.1 μM | IC50 138.6 μM |
| 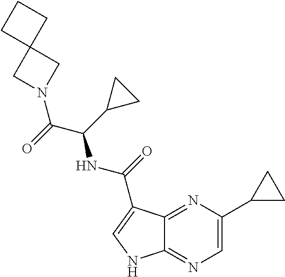 [SMILES: O=C(N[C@@H](C(=O)N1CC2(CCC2)C1)C1CC1)c1c[nH]c2ncc(C3CC3)nc12] | Ki = 15.4 μM | IC50 > 100 μM |
| 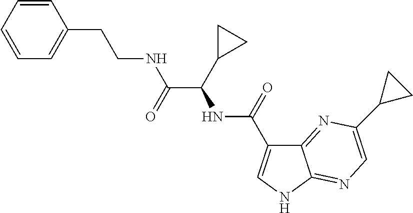 [SMILES: O=C(N[C@@H](C(=O)NCCc1ccccc1)C1CC1)c1c[nH]c2ncc(C3CC3)nc12] | Ki = 16.2 μM | IC50 > 100 μM |
| 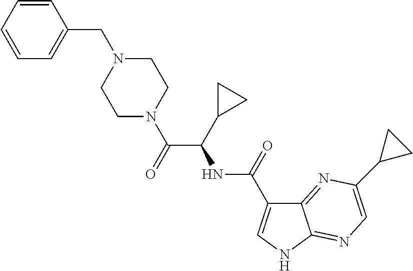 [SMILES: O=C(N[C@@H](C(=O)N1CCN(Cc2ccccc2)CC1)C1CC1)c1c[nH]c2ncc(C3CC3)nc12] | Ki = 165.2 μM | IC50 41.9 μM |
| 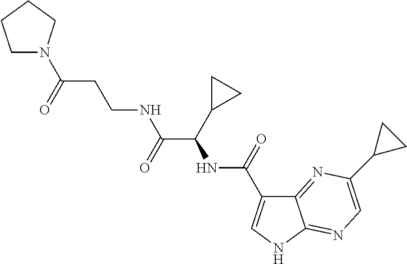 [SMILES: O=C(N[C@@H](C(=O)NCCC(=O)N1CCCC1)C1CC1)c1c[nH]c2ncc(C3CC3)nc12] | Ki = 17.6 μM | IC50 > 100 μM |
| 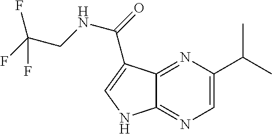 [SMILES: CC(C)c1cnc2[nH]cc(C(=O)NCC(F)(F)F)c2n1] | Ki = 170.5 μM | IC50 > 50 μM |
| 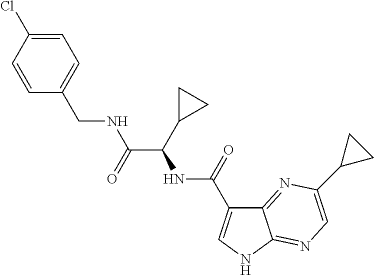 [SMILES: O=C(N[C@@H](C(=O)NCc1ccc(Cl)cc1)C1CC1)c1c[nH]c2ncc(C3CC3)nc12] | Ki = 178.4 μM | IC50 2.99 μM |
| 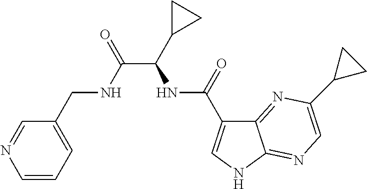 [SMILES: O=C(N[C@@H](C(=O)NCc1cccnc1)C1CC1)c1c[nH]c2ncc(C3CC3)nc12] | Ki = 18.74 μM | 6.6% @ 50 μM |
| 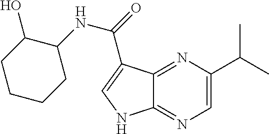 [SMILES: CC(C)c1cnc2[nH]cc(C(=O)NC3CCCCC3O)c2n1] | Ki = 194.9 μM | IC50 11.3 μM |
| 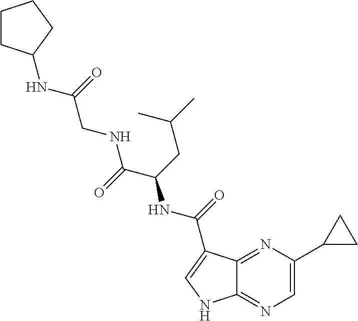 [SMILES: CC(C)C[C@@H](NC(=O)c1c[nH]c2ncc(C3CC3)nc12)C(=O)NCC(=O)NC1CCCC1] | Ki = 2.53 μM | IC50 > 100 μM |
| 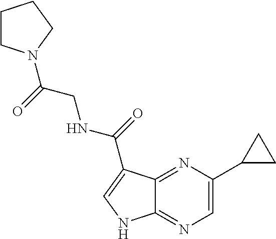 [SMILES: O=C(NCC(=O)N1CCCC1)c1c[nH]c2ncc(C3CC3)nc12] | Ki = 2.7 μM | 36.2% @ 50 μM |
| 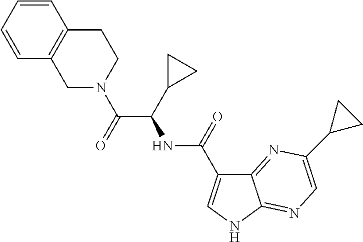 [SMILES: O=C(N[C@@H](C(=O)N1CCc2ccccc2C1)C1CC1)c1c[nH]c2ncc(C3CC3)nc12] | Ki = 20.0 μM | IC50 > 100 μM |
| 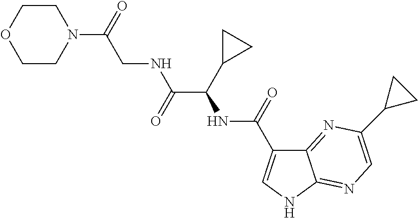 [SMILES: O=C(N[C@@H](C(=O)NCC(=O)N1CCOCC1)C1CC1)c1c[nH]c2ncc(C3CC3)nc12] | Ki = 24.5 μM | IC50 > 100 μM |
| 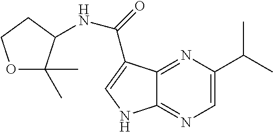 [SMILES: CC(C)c1cnc2[nH]cc(C(=O)NC3CCOC3(C)C)c2n1] | Ki = 240.8 μM | IC50 > 100 μM |
| 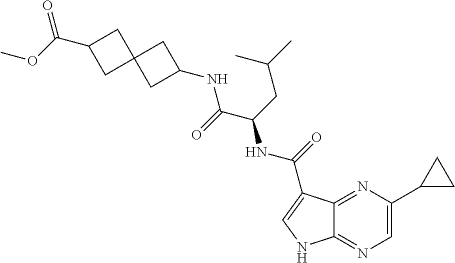 [SMILES: COC(=O)C1CC2(CC(NC(=O)[C@@H](CC(C)C)NC(=O)c3c[nH]c4ncc(C5CC5)nc34)C2)C1] | Ki = 25.0 μM | IC50 > 100 μM |
| 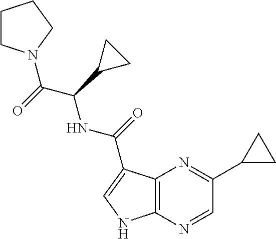 [SMILES: O=C(N[C@@H](C(=O)N1CCCC1)C1CC1)c1c[nH]c2ncc(C3CC3)nc12] | Ki = 26.9 μM | IC50 > 25 μM |
| 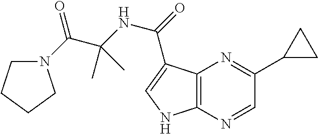 [SMILES: CC(C)(NC(=O)c1c[nH]c2ncc(C3CC3)nc12)C(=O)N1CCCC1] | Ki = 265.2 μM | IC50 > 100 μM |
| 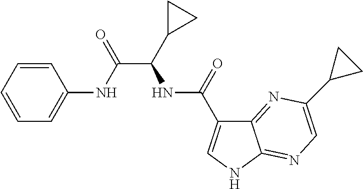 [SMILES: O=C(N[C@@H](C(=O)Nc1ccccc1)C1CC1)c1c[nH]c2ncc(C3CC3)nc12] | Ki = 27.0 μM | IC50 > 100 μM |
| 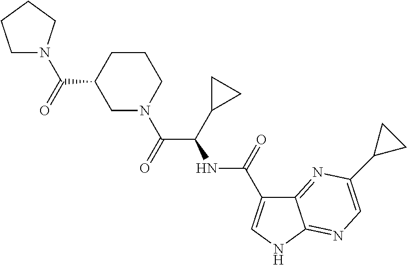 [SMILES: O=C(N[C@@H](C(=O)N1CCC[C@@H](C(=O)N2CCCC2)C1)C1CC1)c1c[nH]c2ncc(C3CC3)nc12] | Ki = 3.5 μM | IC50 > 100 μM |
| 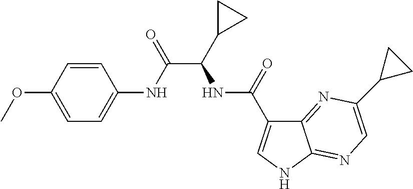 [SMILES: COc1ccc(NC(=O)[C@H](NC(=O)c2c[nH]c3ncc(C4CC4)nc23)C2CC2)cc1] | Ki = 35.9 μM | IC50 > 100 μM |
| 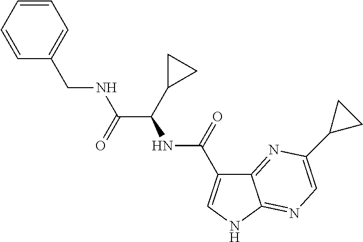 [SMILES: O=C(N[C@@H](C(=O)NCc1ccccc1)C1CC1)c1c[nH]c2ncc(C3CC3)nc12] | Ki = 37.3 μM | IC50 18.2 μM |
| 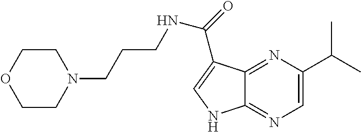 [SMILES: CC(C)c1cnc2[nH]cc(C(=O)NCCCN3CCOCC3)c2n1] | Ki = 37.4 μM | IC50 > 100 μM |
| 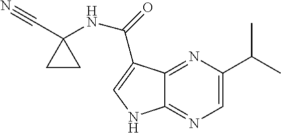 [SMILES: CC(C)c1cnc2[nH]cc(C(=O)NC3(C#N)CC3)c2n1] | Ki = 38.1 μM | IC50 34.7 μM |
| 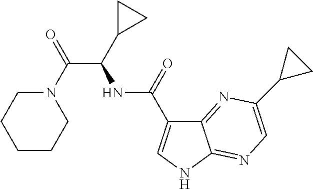 [SMILES: O=C(N[C@@H](C(=O)N1CCCCC1)C1CC1)c1c[nH]c2ncc(C3CC3)nc12] | Ki = 4.4 μM | IC50 > 100 μM |
| 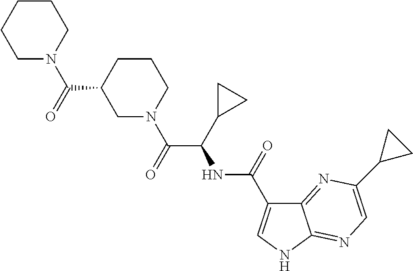 [SMILES: O=C(N[C@@H](C(=O)N1CCC[C@@H](C(=O)N2CCCCC2)C1)C1CC1)c1c[nH]c2ncc(C3CC3)nc12] | Ki = 4.8 μM | IC50 > 100 μM |
| 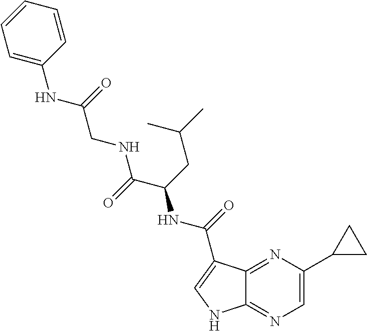 [SMILES: CC(C)C[C@@H](NC(=O)c1c[nH]c2ncc(C3CC3)nc12)C(=O)NCC(=O)Nc1ccccc1] | Ki = 4.87 μM | 25% @ 50 μM |
| 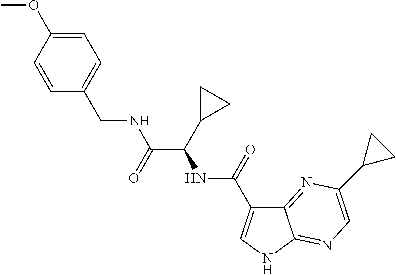 [SMILES: COc1ccc(CNC(=O)[C@H](NC(=O)c2c[nH]c3ncc(C4CC4)nc23)C2CC2)cc1] | Ki = 40.6 μM | IC50 10.3 μM |
| 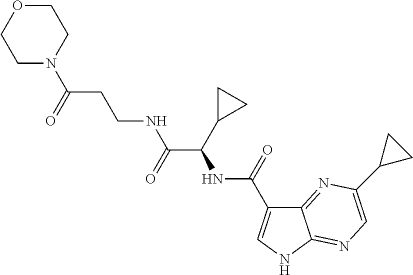 [SMILES: O=C(N[C@@H](C(=O)NCCC(=O)N1CCOCC1)C1CC1)c1c[nH]c2ncc(C3CC3)nc12] | Ki = 45.6 μM | IC50 > 100 μM |
| 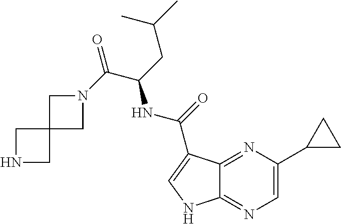 [SMILES: CC(C)C[C@@H](NC(=O)c1c[nH]c2ncc(C3CC3)nc12)C(=O)N1CC2(CNC2)C1] | Ki = 5.13 μM | IC50 > 100 μM |
| 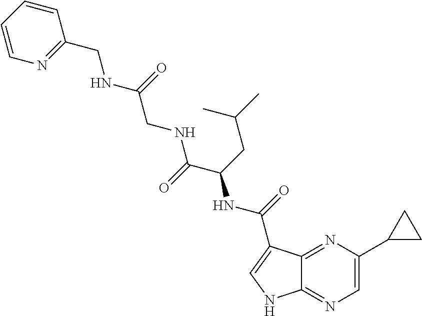 [SMILES: CC(C)C[C@@H](NC(=O)c1c[nH]c2ncc(C3CC3)nc12)C(=O)NCC(=O)NCc1ccccn1] | Ki = 5.53 μM | IC50 > 100 μM |
| 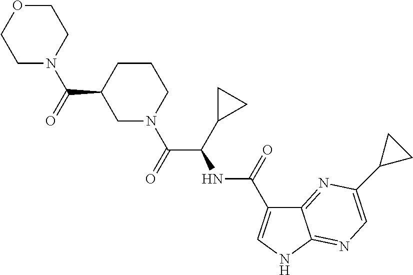 [SMILES: O=C(N[C@@H](C(=O)N1CCC[C@H](C(=O)N2CCOCC2)C1)C1CC1)c1c[nH]c2ncc(C3CC3)nc12] | Ki = 5.7 μM | IC50 > 100 μM |
| 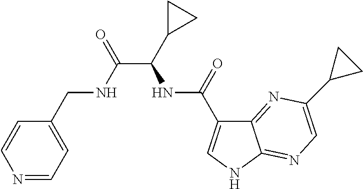 [SMILES: O=C(N[C@@H](C(=O)NCc1ccncc1)C1CC1)c1c[nH]c2ncc(C3CC3)nc12] | Ki = 51.49 μM | IC50 40.4 μM |
| 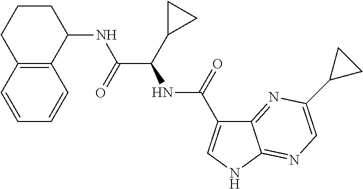 [SMILES: O=C(N[C@@H](C(=O)NC1CCCc2ccccc21)C1CC1)c1c[nH]c2ncc(C3CC3)nc12] | Ki = 53.3 μM | IC50 > 100 μM |
| 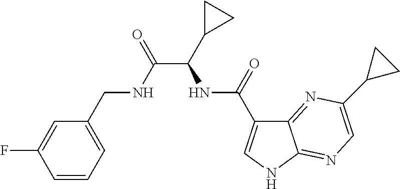 [SMILES: O=C(N[C@@H](C(=O)NCc1cccc(F)c1)C1CC1)c1c[nH]c2ncc(C3CC3)nc12] | Ki = 57.02 μM | IC50 9.2 μM |
| 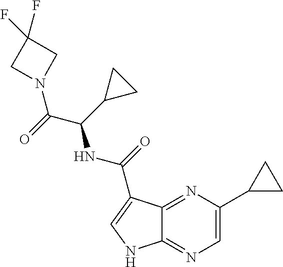 [SMILES: O=C(N[C@@H](C(=O)N1CC(F)(F)C1)C1CC1)c1c[nH]c2ncc(C3CC3)nc12] | Ki = 6.0 μM | IC50 > 100 μM |
| 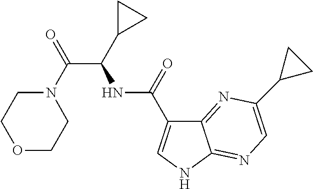 [SMILES: O=C(N[C@@H](C(=O)N1CCOCC1)C1CC1)c1c[nH]c2ncc(C3CC3)nc12] | Ki = 6.2 μM | IC50 > 100 μM |
| 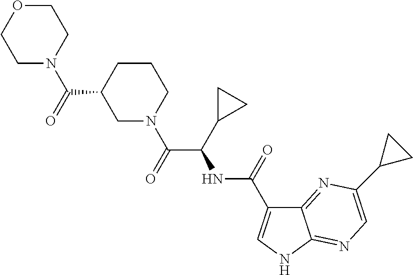 [SMILES: O=C(N[C@@H](C(=O)N1CCC[C@@H](C(=O)N2CCOCC2)C1)C1CC1)c1c[nH]c2ncc(C3CC3)nc12] | Ki = 6.4 μM | IC50 > 100 μM |
| 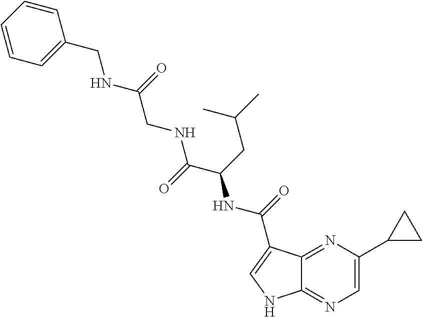 [SMILES: CC(C)C[C@@H](NC(=O)c1c[nH]c2ncc(C3CC3)nc12)C(=O)NCC(=O)NCc1ccccc1] | Ki = 6.82 μM | IC50 > 100 μM |
| 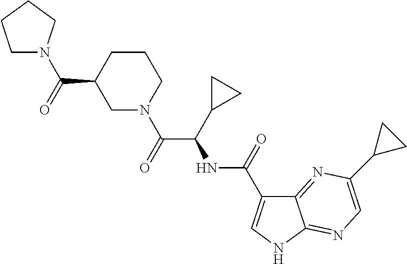 [SMILES: O=C(N[C@@H](C(=O)N1CCC[C@H](C(=O)N2CCCC2)C1)C1CC1)c1c[nH]c2ncc(C3CC3)nc12] | Ki = 6.9 μM | IC50 > 100 μM |
| 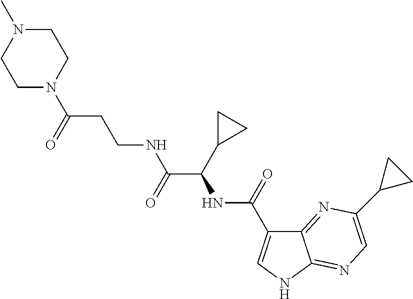 [SMILES: CN1CCN(C(=O)CCNC(=O)[C@H](NC(=O)c2c[nH]c3ncc(C4CC4)nc23)C2CC2)CC1] | Ki = 60.1 μM | IC50 > 100 μM |
| 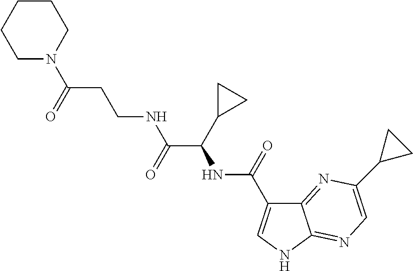 [SMILES: O=C(N[C@@H](C(=O)NCCC(=O)N1CCCCC1)C1CC1)c1c[nH]c2ncc(C3CC3)nc12] | Ki = 63.5 μM | IC50 > 100 μM |
| 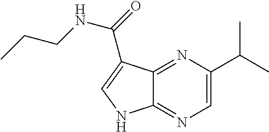 [SMILES: CCCNC(=O)c1c[nH]c2ncc(C(C)C)nc12] | Ki = 65.7 μM | IC50 18.1 μM |
| 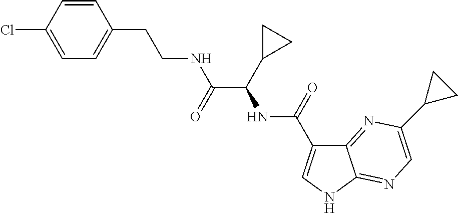 [SMILES: O=C(N[C@@H](C(=O)NCCc1ccc(Cl)cc1)C1CC1)c1c[nH]c2ncc(C3CC3)nc12] | Ki = 68.2 μM | IC50 > 100 μM |
| 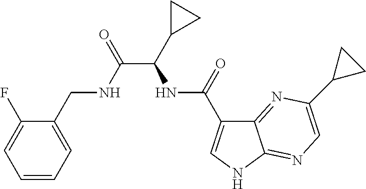 [SMILES: O=C(N[C@@H](C(=O)NCc1ccccc1F)C1CC1)c1c[nH]c2ncc(C3CC3)nc12] | Ki = 68.92 μM | IC50 14.6 μM |
| 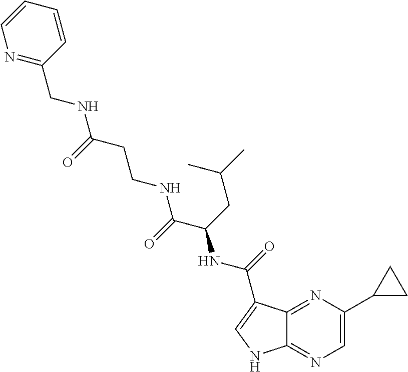 [SMILES: CC(C)C[C@@H](NC(=O)c1c[nH]c2ncc(C3CC3)nc12)C(=O)NCCC(=O)NCc1ccccn1] | Ki = 7.85 μM | IC50 > 100 μM |
| 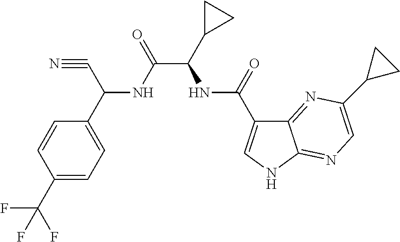 [SMILES: N#CC(NC(=O)[C@H](NC(=O)c1c[nH]c2ncc(C3CC3)nc12)C1CC1)c1ccc(C(F)(F)F)cc1] | Ki = 74.38 μM | IC50 > 10 μM |
| 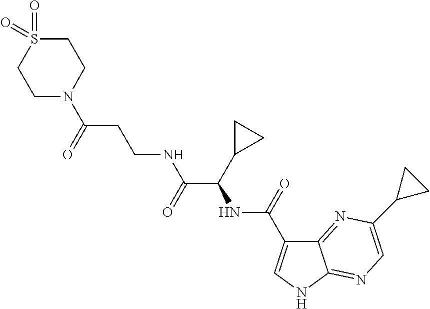 [SMILES: O=C(N[C@@H](C(=O)NCCC(=O)N1CCS(=O)(=O)CC1)C1CC1)c1c[nH]c2ncc(C3CC3)nc12] | Ki = 75.1 μM | IC50 > 100 μM |
| 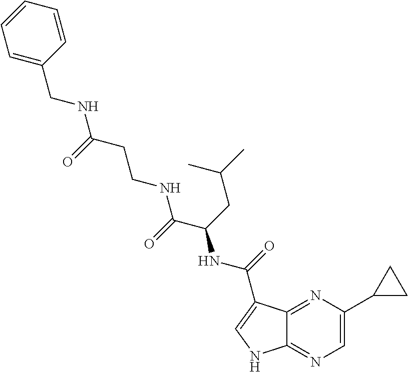 [SMILES: CC(C)C[C@@H](NC(=O)c1c[nH]c2ncc(C3CC3)nc12)C(=O)NCCC(=O)NCc1ccccc1] | Ki = 8.32 μM | IC50 > 100 μM |
| 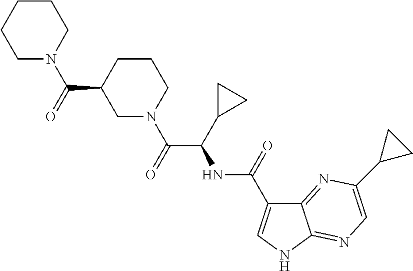 [SMILES: O=C(N[C@@H](C(=O)N1CCC[C@H](C(=O)N2CCCCC2)C1)C1CC1)c1c[nH]c2ncc(C3CC3)nc12] | Ki = 8.5 μM | IC50 33.1 μM |
| 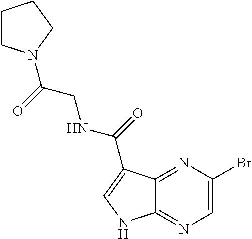 [SMILES: O=C(NCC(=O)N1CCCC1)c1c[nH]c2ncc(Br)nc12] | Ki = 9.2 μM | IC50 > 100 μM |
| 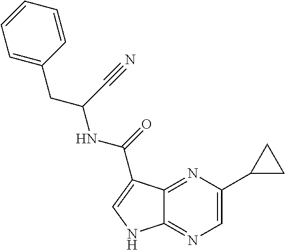 [SMILES: N#CC(Cc1ccccc1)NC(=O)c1c[nH]c2ncc(C3CC3)nc12] | Ki = 9.25 μM | |
| 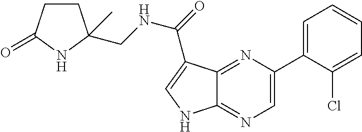 [SMILES: CC1(CNC(=O)c2c[nH]c3ncc(-c4ccccc4Cl)nc23)CCC(=O)N1] | Ki = 907 μM | IC50 > 100 μM |
| 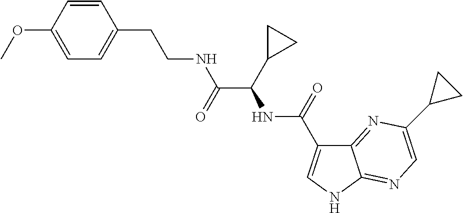 [SMILES: COc1ccc(CCNC(=O)[C@H](NC(=O)c2c[nH]c3ncc(C4CC4)nc23)C2CC2)cc1] | Ki = 98.4 μM | IC50 > 100 μM |
| 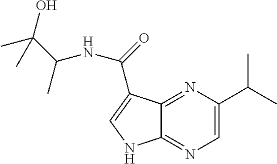 [SMILES: CC(C)c1cnc2[nH]cc(C(=O)NC(C)C(C)(C)O)c2n1] | Ki > 1000 μM | IC50 12.2 μM |
| 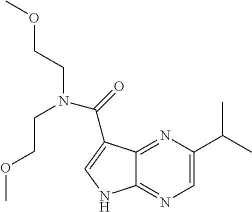 [SMILES: COCCN(CCOC)C(=O)c1c[nH]c2ncc(C(C)C)nc12] | Ki > 1000 μM | IC50 > 100 μM |
| 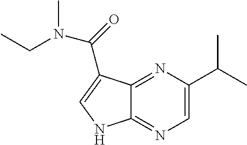 [SMILES: CCN(C)C(=O)c1c[nH]c2ncc(C(C)C)nc12] | Ki > 1000 μM | IC50 > 100 μM |
| 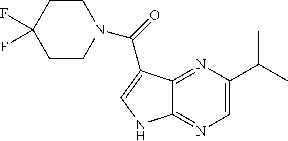 [SMILES: CC(C)c1cnc2[nH]cc(C(=O)N3CCC(F)(F)CC3)c2n1] | Ki > 1000 μM | IC50 > 100 μM |
| 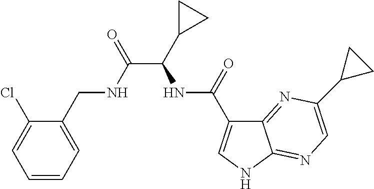 [SMILES: O=C(N[C@@H](C(=O)NCc1ccccc1Cl)C1CC1)c1c[nH]c2ncc(C3CC3)nc12] | Ki > 125 μM | IC50 8.8 μM |
| 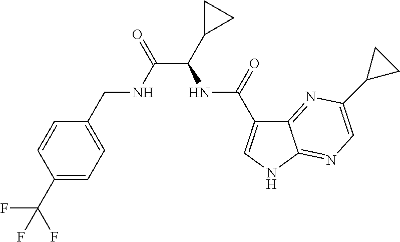 [SMILES: O=C(N[C@@H](C(=O)NCc1ccc(C(F)(F)F)cc1)C1CC1)c1c[nH]c2ncc(C3CC3)nc12] | Ki > 125 μM | IC50 4.6 μM |
| 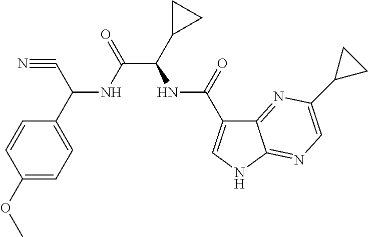 [SMILES: COc1ccc(C(C#N)NC(=O)[C@H](NC(=O)c2c[nH]c3ncc(C4CC4)nc23)C2CC2)cc1] | Ki > 125 μM | IC50 4.9 μM |
| 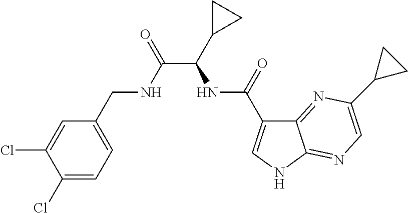 [SMILES: O=C(N[C@@H](C(=O)NCc1ccc(Cl)c(Cl)c1)C1CC1)c1c[nH]c2ncc(C3CC3)nc12] | Ki > 125 μM | IC50 4.7 μM |
| 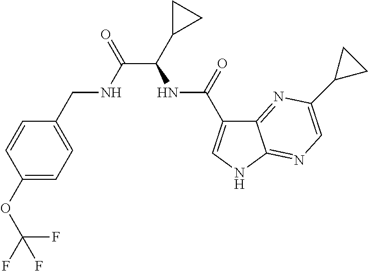 [SMILES: O=C(N[C@@H](C(=O)NCc1ccc(OC(F)(F)F)cc1)C1CC1)c1c[nH]c2ncc(C3CC3)nc12] | Ki > 125 μM | IC50 5.4 μM |
| 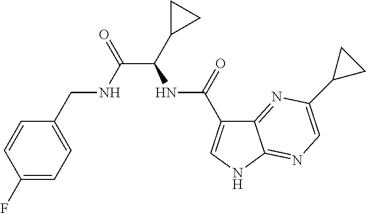 [SMILES: O=C(N[C@@H](C(=O)NCc1ccc(F)cc1)C1CC1)c1c[nH]c2ncc(C3CC3)nc12] | Ki > 31.25 μM | IC50 16.1 μM |
| 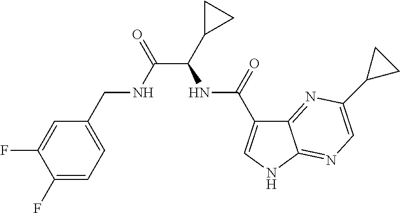 [SMILES: O=C(N[C@@H](C(=O)NCc1ccc(F)c(F)c1)C1CC1)c1c[nH]c2ncc(C3CC3)nc12] | Ki > 31.25 μM | IC50 14.8 μM |
| 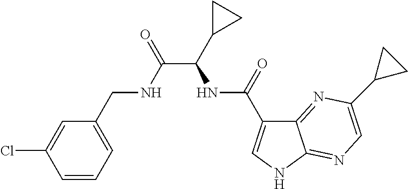 [SMILES: O=C(N[C@@H](C(=O)NCc1cccc(Cl)c1)C1CC1)c1c[nH]c2ncc(C3CC3)nc12] | Ki > 62.5 μM | IC50 7.3 μM |
| 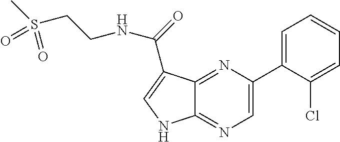 [SMILES: CS(=O)(=O)CCNC(=O)c1c[nH]c2ncc(-c3ccccc3Cl)nc12] | Ki > 655 μM | IC50 > 100 μM |
| 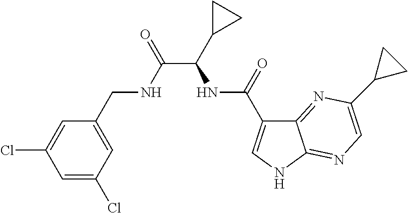 [SMILES: O=C(N[C@@H](C(=O)NCc1cc(Cl)cc(Cl)c1)C1CC1)c1c[nH]c2ncc(C3CC3)nc12] | Ki > 7.8 μM | IC50 > 25 μM |
| 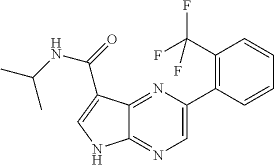 [SMILES: CC(C)NC(=O)c1c[nH]c2ncc(-c3ccccc3C(F)(F)F)nc12] | Ki ≧ 62.5 μM | IC50 > 100 μM |
| 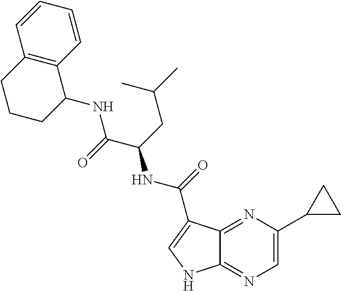 [SMILES: CC(C)C[C@@H](NC(=O)c1c[nH]c2ncc(C3CC3)nc12)C(=O)NC1CCCc2ccccc21] | Ki ≧ 125 μM | IC50 > 100 μM |
| 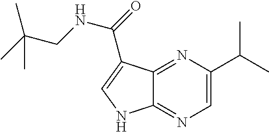 [SMILES: CC(C)c1cnc2[nH]cc(C(=O)NCC(C)(C)C)c2n1] | No displacement | IC50 52.2 μM |
| 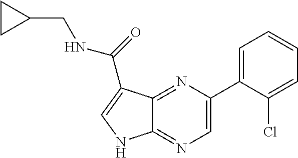 [SMILES: O=C(NCC1CC1)c1c[nH]c2ncc(-c3ccccc3Cl)nc12] | No displacement | |
| 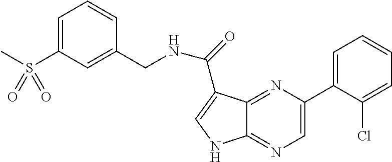 [SMILES: CS(=O)(=O)c1cccc(CNC(=O)c2c[nH]c3ncc(-c4ccccc4Cl)nc23)c1] | No displacement | IC50 22.5 |

1. A method of treating, ameliorating or preventing influenza, wherein an effective amount of a compound having the general formula (I), optionally in the form of a pharmaceutically acceptable salt, solvate, polymorph, prodrug, codrug, cocrystal, tautomer, racemate, codrug, cocrystal, enantiomer, or diastereomer or mixture thereof 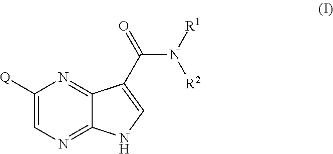
wherein
Q is an organic substituent;
R¹ is an organic substituent; and
R² is an organic substituent;
is administered to a patient in need thereof.

2. The method according to claim 1, wherein
Q is Q¹, Q², or Q³;
Q¹ is cycloalkyl, heterocycloalkyl, cycloalkyloxy, cycloalkenyl, heterocycloalkyl aryl, aryloxy, heteroaryl, biaryl, or heterobiaryl, optionally substituted with one or more Q^{1a};
Q^{1a} is Q^{1b} or Q^{1c};
each Q^{1b} is independently halogen, oxo, hydroxy, cyano, —SCH₃, —S(O)₂CH₃, or —S(═O)CH₃;
each Q^{1c} is independently Q^{1d} or Q^{1e};
or two Q^{1a} come together to form a bicyclic ring system, optionally substituted with one or more Q^{1b} or Q^{1c};
each Q^{1d} is independently —O(Q^{1e}), —S(═O)₂(Q^{1e}), —C(═O)N(Q^{1e})₂, —S(O)₂(Q^{1e}), —C(═O)(Q^{1e}), —C(═O)O(Q^{1e}), —N(Q^{1e})₂, —N(Q^{1e})C(═O)(Q^{1e})-N(Q^{1e})C(═O)O(Q^{1e}), or —N(Q^{1e})C(═O)N(Q^{1e})₂;
each Q^{1e} is independently H or Q^{1j};
each Q^{1f} is independently Q^{1g} or Q^{1h};
each Q^{1g} is independently halogen, hydroxy, cyano, oxo, —C(═O)(Q^{1h}), —S(═O)₂(Q^{1k}), —S(═O)₂N(Q^{1k})₂, —C(═O)OH, C(═O)N(Q^{1k})₂, or —C(═O)(Q^{1k});
each Q^{1h} is independently lower alkyl, lower alkenyl, lower haloalkyl, lower alkoxy, amino, aryl, benzyl, cycloalkyl, heterocycloalkyl, or heteroaryl, optionally substituted with one or more Q¹ⁱ;
each Q¹ⁱ is independently halogen, hydroxy, cyano, lower alkyl, lower haloalkyl, or lower alkoxy;
each Q^{1j} is independently lower alkyl, aryl, benzyl, 5,6,7,8-tetrahydro-naphthalene, lower haloalkyl, lower alkoxy, cycloalkyl, cycloalkyl lower alkyl, cycloalkenyl, heterocycloalkyl, spirocyclic heterocycloalkyl, or heteroaryl, optionally substituted with one or more Q^{1f};
each Q^{1k} is independently H or lower alkyl;
Q² is Q^{2a} or Q^{2b};
Q^{2a} is H, hydroxy, halogen, or cyano;
Q^{2b} is lower alkyl, lower alkoxy, lower alkenyl, lower alkynyl, lower hydroxyalkyl, amino, or lower haloalkyl, optionally substituted with one or more Q^{2c};
Q^{2c} is Q^{2d} or Q^{2e};
Q^{2d} is halogen, oxo, hydroxy, cyano, —C(═O)(Q^{2j}), —SCH₃, —S(O)₂CH₃, or —S(═O)CH₃;
Q^{2e} is Q^{2f} or Q^{2j};
or two Q^{2c} come together to form a bicyclic ring system, optionally substituted with one or more Q^{2d} or Q^{2e};
Q^{2f} is —O(Q^{2g}), —S(═O)₂(Q^{2g}), —C(═O)N(Q^{2g})₂, —S(O)₂(Q^{2g}), —C(═O)(Q^{2g}), —C(═O)O(Q^{2g}), —N(Q^{2g})₂; —N(Q^{2g})C(═O)(Q^{2g})-N(Q^{2g})C(═O)O(Q^{2g}), or —N(Q^{2g})C(═O)N(Q^{2g})₂;
each Q^{2g} is independently H or Q^{2m};
Q^{2h} is Q²ⁱ or Q^{2j};
Q²ⁱ is halogen, hydroxy, cyano, oxo, or —C(═O)(Q^{2j});
Q^{2j} is lower alkyl, lower alkenyl, lower alkoxy, amino, aryl, benzyl, cycloalkyl, heterocycloalkyl, or heteroaryl, optionally substituted with one or more Q^{2k};
Q^{2k} is halogen, hydroxy, cyano, lower alkyl, lower haloalkyl, lower alkenyl, oxo, lower hydroxyalkyl, amino or lower alkoxy;
each Q^{2m} is independently lower alkyl, aryl, benzyl, lower haloalkyl, lower alkoxy, amino, cycloalkyl, cycloalkyl lower alkyl, cycloalkenyl, heterocycloalkyl, or heteroaryl, optionally substituted with one or more Q^{2h};
Q³ is aryl or heteroaryl, optionally substituted with one or more Q^{3a};
each Q^{3a} is independently Q^{3b} or Q^{3c};
each Q^{3b} is independently halogen, hydroxy, cyano, —S(Q^{3e}), —S(O)₂(Q^{3e}), or —S(═O)(Q^{3e});
each Q^{3c} is independently Q^{3d} or Q^{3e};
each Q^{3d} is independently —O(Q^{3e}), —S(═O)₂(Q^{3e}), —C(═O)N(Q^{3e})₂, —S(═O)(Q^{3e}), —N(Q^{3e})S(═O)₂(Q^{3e}), —C(═O)(Q^{3e}), —C(═O)O(Q^{3e}), —N(Q^{3e})₂, —N(Q^{3e})C(═O)(Q^{3e}), —N(Q^{3e})C(═)O(Q^{3e}), —Si(Q^{3e})₃, or —N(Q^{3e})C(═O)N(Q^{3e})₂;
each Q^{3e} is independently H or Q^{3m};
each Q^{3f} is independently Q^{3g} or Q^{3h};
each Q^{3g} is independently halogen, hydroxy, oxo, —(C(Q^{3h})₂)_{mQ}S(O)₂(Q^{3h}), —(C(Q^{3h})₂)_{mQ}N(Q^{3h})(C(Q^{3h})₂)_{mQ}S(O)₂(Q^{3h}), —(C(Q^{3h})₂)_{mQ}N(Q^{3h})₂, —(C(Q^{3h})₂)_{mQ}C(═O)(Q^{3h}), or —N(Q^{3h})C(═O)(Q^{3h});
each Q^{3h} is independently Q³ⁱ or Q^{3j};
each Q³ⁱ is independently H or hydroxy;
each Q^{3j} is independently lower alkyl, lower haloalkyl, lower alkoxy, lower thioalkyl, cyano, amino, aryl, benzyl, cycloalkyl, heterocycloalkyl, or heteroaryl, optionally substituted with one or more Q^{3k};
each Q^{3k} is independently halogen, hydroxy, lower alkyl, lower haloalkyl, lower hydroxyalkyl, amino, lower thioalkyl, lower alkoxy, or cyano;
each Q^{3m} is independently lower alkyl, amino, lower alkenyl, aryl, benzyl, lower haloalkyl, lower thioalkyl, cycloalkyl, cycloalkenyl, heterocycloalkyl, heterocycloalkyl alkylene, or heteroaryl, optionally substituted with one or more Q^{3f};
each m_{Q} is independently 0, 1, or 2.

3. The method according to claim 2, wherein Q is selected from the group consisting of cycloalkyl, halogen, lower alkyl and aryl which is optionally substituted with one or more Q^{3a}, wherein Q^{3a} is selected from the group consisting of halogen, haloalkyl, cycloalkyl-C(O)—OH and cycloalkyl-C(O)—O-(lower alkyl).

4. The method according to claim 3, wherein Q is selected from the group consisting of cycloalkyl and aryl which is optionally substituted with cycloalkyl-C(O)—OH or cycloalkyl-C(O)—O-(lower alkyl).

5. The method according to claim 3, wherein Q is cyclopropyl.

6. The method according to claim 1, wherein R¹ and R² are selected from (i) to (v):
(i) R¹ is H and
R² is —Y—C(O)—NR^{1e}R^{1g};
Y is C(R^{1a})₂(C(R^{1b})₂)m_{R};
m_{R} is 0 or 1;
each R^{1a} is H or R^{1c};
each R^{1b} is independently H, lower alkyl, lower haloalkyl, aryl, heteroaryl, cycloalkyl, heterocycloalkyl, wherein the aryl, heteroaryl, cycloalkyl, heterocycloalkyl can be optionally substituted by H, halogen, lower alkyl, lower alkoxy, or lower haloalkyl;
each R^{1c} is independently lower alkyl, lower alkoxy, aryl, benzyl, heteroaryl, cycloalkyl, heterocycloalkyl, or cycloalkyl lower alkyl, optionally substituted with one or more R^{1d};
R^{1d} is independently R^{1j} or R^{1k};
R^{1e} is independently H or R^{1f};
R^{1f} is independently lower alkyl, lower alkoxy, aryl, heteroaryl, cycloalkyl, heterocycloalkyl, bicyclic ring system or spirocyclic ring system, wherein the bicyclic ring system or spirocyclic ring system can optionally include one or more heteroatoms or heteroatom containing moieties such as C═O, wherein R^{1f} can be optionally substituted with one or more R^{1d};
or R^{1f} and R^{1c} come together to form a ring, optionally substituted with one or more one or more halogen, lower alkyl, cyano, cyano lower alkyl, hydroxy, lower haloalkyl, lower hydroxyalkyl, lower alkoxy, lower alkylamino, or lower dialkylamino;
R^{1g} is independently H or R^{1h};
R^{1h} is independently lower alkyl, lower haloalkyl, lower alkoxy, lower hydroxyalkyl, cyano lower alkyl, C(═O)R¹ⁱ or S(═O)₂R¹ⁱ;
each R¹ⁱ is independently H or lower alkyl;
R^{1j} is independently halogen, lower alkyl, lower haloalkyl, lower alkoxy, lower haloalkoxy, lower hydroxyalkyl, oxo, hydroxy, C(═O)—NH—(CH₂)ₙ₁—R^{1b}, C(═O)—(CH₂)ₙ₁—R^{1b}, (C═O)—OR^{1b} or cyano;
R^{1k} is independently —(CH₂)ₙ₁-cycloalkyl, —(CH₂)ₙ₁-heterocycloalkyl, —(CH₂)ₙ₁-aryl, —(CH₂)ₙ₁-heteroaryl, optionally substituted by halogen, lower alkyl, lower haloalkyl, lower alkoxy, lower haloalkoxy, lower hydroxyalkyl, hydroxy, C(═O)—R^{1b}, (C═O)—OR^{1b}, C(═O)—NH—R^{1b}, C(═O)—NH—CH₂—R^{1b}, or cyano; and
n₁ is 0 or 1;
(ii) R¹ and R² are independently H or R^{2b};
each R^{2b} is independently lower alkyl, lower alkoxy, aryl, heteroaryl, cycloalkyl, heterocycloalkyl, or heterocycloalkyl alkylene, optionally substituted with one or more R^{2c};
R^{2c} is R^{2d} or R^{2e};
each R^{2d} is independently halogen, cyano, oxo, or hydroxy;
each R^{2e} is independently —OR^{2g}, —N(R^{2g})₂, —C(═O)(R^{2g}), —C(═O)O(R^{2g}), —C(═O)N(R^{2g})₂, —N(R^{2g})C(═O)(R^{2g}), —S(═O)₂(R^{2g}), —S(O)₂N(R^{2g})₂, lower alkyl, lower alkoxy, lower haloalkyl, aryl, heteroaryl, heteroaryloxy, cycloalkyl, or heterocycloalkyl, optionally substituted with one or more R^{2f};
each R^{2f} is independently H, halogen, lower alkyl, lower alkoxy, oxo, or lower haloalkyl; and
each R^{2g} is independently H, lower alkyl, lower alkoxy, lower haloalkyl, or aryl;
(iii) R¹ is H and
R² is 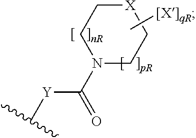
X is C(R^{3d})(R^{3e}), N(R^{3d}), S(═O)₂, or O;
each X′ is independently halogen, lower alkyl, cyano, hydroxy, C(═O)—OR^{3g}, C(═O)R^{3g}, lower haloalkyl, lower hydroxyalkyl, heteroaryl, spiroheterocycloalkyl, spirocycloalkyl, lower alkoxy, lower alkylamino, or lower dialkylamino;
or two adjacent X′ come together to form a ring which can be saturated or unsaturated;
Y is C(R^{3a})₂(C(R³ⁱ)₂)m_{R};
R^{3a} is independently H or R^{3b};
R^{3b} is lower alkyl, lower alkoxy, aryl, benzyl, heteroaryl, cycloalkyl, heterocycloalkyl, or cycloalkylalkyl, optionally substituted with one or more R^{3c};
R^{3c} is halogen, lower alkyl, lower haloalkyl, lower alkoxy, lower hydroxyalkyl, lower haloalkyl, oxo, hydroxy, or cyano;
each R^{3d} is independently H or R^{3f};
R^{3e} is H, hydroxy, halogen or lower alkyl;
or R^{3d} and R^{3e} come together to form a spirocyclic ring system, wherein the spirocyclic ring system can optionally include one or more heteroatoms or heteroatom containing moieties such as C═O and wherein the spirocyclic ring system can be optionally substituted with one or more R^{3h};
or X′ and R^{3d} come together to form a bicyclic ring system, optionally substituted with one or more R^{3h};
each R^{3f} is independently lower alkyl, lower haloalkyl, halogen, lower alkoxy, lower hydroxyalkyl, cycloalkyl, heterocycloalkyl, aryl, heteroaryl, lower alkylene-cycloalkyl, lower alkylene-heterocycloalkyl, lower alkylene-aryl, lower alkylene-heteroaryl, cyano, cyano lower alkyl, hydroxy, C(═O)—OR^{3g}, C(═O)R^{3g} or S(═O)₂R^{3g};
each R^{3g} is independently H, OR³ⁱ, aryl, heteroaryl, lower alkyl, cycloalkyl or heterocycloalkyl;
R^{3h} is halogen, lower alkyl, lower alkoxy, hydroxy, hydroxy lower alkyl, lower haloalkyl, lower hydroxyalkylcyano, cycloalkyl, heterocycloalkyl, aryl, heteroaryl, lower alkylene-cycloalkyl, lower alkylene-heterocycloalkyl, lower alkylene-aryl, lower alkylene-heteroaryl, —C(O)O—R^{3g} or —S(O)₂CH₃;
each R³ⁱ is independently H, lower alkyl, or lower haloalkyl;
m_{R} is 0 or 1;
n_{R} is 0 or 1;
P_{R} is 0 or 1; and
q_{R} is 0, 1, 2, 3, or 4;
(iv) R¹ is H or OH;
R² is aryl, heterocycloalkyl, heteroaryl or cycloalkyl, each optionally substituted with one or more R^{4a};
each R^{4a} is independently hydroxy, halo, oxo, lower alkyl, lower alkenyl, lower alkynyl, lower haloalkyl, lower alkoxy, lower haloalkoxy, lower hydroxyalkyl, amino, lower alkylamino, lower dialkylamino, cyano, lower cyanoalkyl, cycloalkyl, heterocycloalkyl, C(═O)R^{4b}, or S(═O)₂R^{4b}; and
each R^{4b} is independently OH, cycloalkyl or lower alkyl;
(v) R¹ is H;
R² is lower alkoxy or 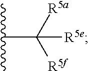
or R¹ and R² together form heterocycloalkyl, optionally substituted with halogen or cyano;
R^{5a} is H, cyano, lower alkyl, R^{5b}, R^{5q or} 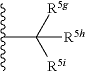
R^{5b} is cycloalkyl, heterocycloalkyl, heteroaryl, or aryl, wherein each is optionally substituted with one or more R^{5c};
each R^{5c} is independently halo, hydroxy, cyano, lower alkyl, lower haloalkyl, lower alkoxy, lower hydroxyalkyl, cycloalkyl, C(═O)R^{5d}, or S(═O)₂R^{5d};
each R^{5d} is independently OH or lower alkyl;
R^{5e} is H, hydroxy lower alkyl, lower haloalkyl, or lower alkyl;
R^{5f} is H, hydroxy, cyano, cyano lower alkyl, —C(═O)NH₂, —C(═O)OH, —C(═O)OC(CH₃)₃, R^{5r}, R^{5s} or R^{5k};
R^{5g} and R^{5h} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, C(═O)R^{5d}, S(═O)₂R^{5d} or CH₂S(═O)₂R^{5d};
R⁵ⁱ is aryl, cycloalkyl, heterocycloalkyl, or heteroaryl, optionally substituted with one or more R^{5j};
each R^{5j} is independently hydroxy, halo, lower alkyl, lower hydroxyalkyl, lower halo alkyl, or lower alkoxy;
each R^{5k} is independently lower alkyl, hydroxy lower alkyl, lower alkoxy, lower haloalkyl, lower haloalkoxy, aryl lower alkyl, cycloalkyl or cycloalkyl lower alkyl, each optionally substituted with one or more R^{5m};
each R^{5m} is independently lower alkyl, halo, hydroxy, lower alkoxy, lower haloalkyl, lower hydroxy alkyl, oxo, amino, cyano, cyano lower alkyl, S(═O)₂R⁵ⁿ, C(═O)R⁵ⁿ, cycloalkyl, heterocycloalkyl, heteroaryl, lower alkyl sulfonylamino, lower alkyl sulfonyl, halo lower alkoxy, cycloalkyl, —C(═O)OCH₃ or heterocycloalkenyl;
each R⁵ⁿ is independently H, hydroxy or lower alkyl;
each R^{5p} is independently hydroxy, amino, oxo, lower alkyl, —C(═O)NH₂, cyano, lower haloalkyl, benzyl, cyano lower alkyl, or —NHC(═O)OC(CH₃)₃;
R^{5q} is lower alkoxyl, hydroxy lower alkyl, or lower haloalkyl;
or R^{5q} and R^{5e} together form heterocycloalkyl, cycloalkyl, indan-1-yl, aryl, or heteroaryl, optionally substituted with one or more R^{5p};
R^{5r} is aryl, heteroaryl, heterocycloalkyl, heterocycloalkyl lower alkyl, heteroaryl lower alkyl, aryl lower alkoxy, optionally substituted with one or more R^{5m};
R^{5s} is —C(═O)R^{5t} or —CH₂C(═O)R^{5t};
R^{5t} is heterocycloalkyl, optionally substituted with one or more R^{5u}; and
each R^{5u} is independently cyano, halo, lower alkyl, or lower alkyl sulfonyl.

7. The method according to claim 6, wherein R¹ and R² are as defined in option (i), (ii) or (iii).

8. The method according to claim 6, wherein
R¹ is H;
R² is —CHR^{1a}—C(O)—NR^{1e}R^{1g};
R^{1a} is cycloalkyl (preferably cyclopropyl), H, or lower alkyl;
R^{1d} is cyano, —(CH₂)ₙ₁—R**, C(O)—(CH₂)ₙ₁—R** or C(O)—NH—(CH₂)ₙ₁—R**, wherein R** is optionally substituted with one or more of halogen, lower haloalkyl, (C═O)—OR*, lower alkyl, lower alkoxy, lower haloalkoxy, or cyano;
R^{1e} is H, cycloalkyl, aryl or lower alkyl, wherein cycloalkyl, aryl or lower alkyl can be optionally substituted with one or more R^{1d};
R^{1g} is H;
R* is H or lower alkyl;
R** is cycloalkyl, aryl, heterocycloalkyl or heteroaryl; and
n₁ is 0 or 1.

9. The method according to claim 6, wherein
R¹ is H;
R² is lower alkyl, aryl, heterocycloalkyl, heteroaryl or cycloalkyl, wherein lower alkyl, aryl, heterocycloalkyl, heteroaryl or cycloalkyl can be optionally substituted with one or more R^{2c};
R^{2c} is cycloalkyl, heterocycloalkyl or heteroaryl, aryl, OR*, COOR*, halogen, cyano or —S(O)₂—R*, wherein cycloalkyl, heterocycloalkyl, heteroaryl and aryl can be optionally substituted by lower alkyl, lower alkoxy, or lower haloalkyl; more preferably R^{2c} is heteroaryl, aryl, cyano, COOR* or —S(O)₂—R*, wherein heteroaryl and aryl can be optionally substituted by lower alkyl, lower alkoxy, or lower haloalkyl; and; and
R* is H or lower alkyl.

10. The method according to claim 6, wherein
R¹ is H;
R² is O 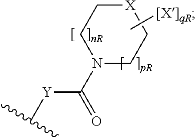
X′ is halogen, hydroxy, lower hydroxyalkyl, C(O)OR^{3g} or C(O)R^{3g};
or adjacent X′ come together to form a ring which can be saturated or unsaturated;
Y is CH(R^{3b});
n_{R} is 0 or 1;
p_{R} is 0 or 1;
q_{R} is 0 or 1;
R^{3b} is H, cycloalkyl or lower alkyl;
R^{3g} is OR*, cycloalkyl, aryl, heterocycloalkyl, or heteroaryl
R* is H or lower alkyl;
X is CF₂, CH₂, O, or N(R^{3d}) in which R^{3d} is lower alkylene-aryl, heterocycloalkyl;
or X is C(R^{3d})(R^{3e}) in which R^{3d} and R^{3e} come together to form a spirocyclic ring system which can optionally include one or more heteroatoms or heteroatom containing moieties and wherein the spirocyclic ring system can be optionally substituted with one or more R^{3h} such as benzyl or —C(O)O—R*.

11. The method according to claim 6, wherein
(vi) R₁ is H;
R² is R 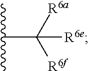
R^{6a} is H, cyano, lower alkyl, R^{6b} or, 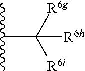
R^{6b} is cycloalkyl, heterocycloalkyl, heteroaryl, or aryl, wherein each is optionally substituted with one or more R^{6c};
each R^{6c} is independently halo, hydroxy, cyano, lower alkyl, lower haloalkyl, lower alkoxy, lower hydroxyalkyl, cycloalkyl, C(═O)R^{6d}, or S(═O)₂R^{6d};
each R^{6d} is independently OH or lower alkyl;
R^{6e} is H, hydroxy lower alkyl, lower haloalkyl, or lower alkyl;
R^{6f} is H, hydroxy, cyano, cyano lower alkyl, or R^{6k};
R^{6g} and R^{6h} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, C(═O)R^{6d}, S(═O)₂R^{6d} or CH₂S(═O)₂R^{6d};
R⁶ⁱ is aryl, cycloalkyl, heterocycloalkyl, or heteroaryl, optionally substituted with one or more R^{6j};
each R^{6j} is independently hydroxy, halo, lower alkyl, lower hydroxyalkyl, lower halo alkyl, or lower alkoxy;
each R^{6k} is independently lower alkyl, hydroxy lower alkyl, lower alkoxy, lower haloalkyl, lower haloalkoxy, aryl lower alkyl, cycloalkyl or cycloalkyl lower alkyl, each optionally substituted with one or more R^{6m};
each R^{6m} is independently lower alkyl, halo, hydroxy, lower alkoxy, lower haloalkyl, lower hydroxy alkyl, oxo, amino, cyano, cyano lower alkyl, S(═O)₂R⁶ⁿ, C(═O)R⁶ⁿ, cycloalkyl, heterocycloalkyl, heteroaryl, or heterocycloalkenyl; and
each R⁶ⁿ is independently H, hydroxy or lower alkyl.

12. The method according to claim 6, wherein
(vii) R¹ is H;
R² is lower alkoxy or 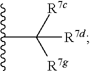
or R¹ and R² together form heterocycloalkyl, optionally substituted with halogen or cyano;
R^{7c} is H or R^{7f};
R^{7d} is H or lower alkyl;
each R^{7e} is independently hydroxy, amino, oxo, lower alkyl, —C(═O)NH₂, cyano, lower haloalkyl, benzyl, cyano lower alkyl, or —NHC(═O)OC(CH₃)₃;
R^{7f} is lower alkyl, cycloalkyl, lower alkoxyl, hydroxy lower alkyl, or lower haloalkyl;
or R^{7f} and R^{7d} together form heterocycloalkyl, cycloalkyl, indan-1-yl, aryl, or heteroaryl, optionally substituted with one or more R^{7e};
R^{7g} is H, hydroxy, cyano, —C(═O)NH₂, —C(═O)OH, —C(═O)OC(CH₃)₃, R^{7h}, or R^{7j};
R^{7h} is lower alkyl, aryl, aryl lower alkyl, cycloalkyl, heteroaryl, heterocycloalkyl, heterocycloalkyl lower alkyl, heteroaryl lower alkyl, aryl lower alkoxy, lower alkoxy, optionally substituted with one or more R⁷ⁱ;
each R⁷ⁱ is independently hydroxy, cyano, amino, lower alkyl sulfonylamino, lower alkoxy, halo, lower alkyl, cyano lower alkyl, lower haloalkyl, lower alkyl sulfonyl, oxo, halo lower alkoxy, cycloalkyl, —C(═O)OCH₃;
R^{7j} is —C(═O)R^{7k} or —CH₂C(═O)R^{7k};
R^{7k} is heterocycloalkyl, optionally substituted with one or more R^{7m}; and
each R^{7m} is independently cyano, halo, lower alkyl, or lower alkyl sulfonyl.

13. The method according to claim 1, wherein at least one further pharmaceutically active agent is to be administered concurrently or sequentially with the compound having the general formula (I) and wherein the at least one further pharmaceutically active agent is selected from the group consisting of:
(i) a polymerase inhibitor which is different from the compound having the general formula (I);
(ii) a neuramidase inhibitor;
(iii) a M2 channel inhibitor;
(iv) an alpha glucosidase inhibitor;
(v) a ligand of another influenza target; and
(vi) a medicament selected from antibiotics, anti-inflammatory agents, lipoxygenase inhibitors, EP ligands, bradykinin ligands, and cannabinoid ligands.
